(19) United States
(12) Patent Application Publication
(10) Pub. No.: US 2013/0261183 A1
(43) Pub. Date: Oct. 3, 2013

(54) OPTIMIZED NUTRITIONAL FORMULATIONS, METHODS FOR SELECTION OF TAILORED DIETS THEREFROM, AND METHODS OF USE THEREOF

(71) Applicant: Urvashi Bhagat, California, CA (US)

(72) Inventor: Urvashi Bhagat, California, CA (US)

(21) Appl. No.: 13/877,847

(22) PCT Filed: Oct. 14, 2011
(86) PCT No.: PCT/US11/56463

§ 371 Date: Apr. 4, 2013

Related U.S. Application Data
(60) Provisional application No. 61/393,235, filed on Oct. 14, 2010.
(60) Provisional application No. 61/415,096, filed on Nov. 18, 2010.

(51) Int. Cl. A23L 1/29; A61K 31/202

(52) U.S. Cl. CPC A23L 1/296; A61K 31/202; A23L 1/293
USPC 514/560; 426/648; 426/72; 426/61; 426/71; 426/599; 426/618; 426/634; 426/632; 426/629; 426/638; 426/641; 426/643; 426/615; 426/580; 426/583; 426/87; 434/127

(57) ABSTRACT

Nutritional compositions and formulations that optimize nutritional contents are provided. Dietary compositions and methods for tailoring such compositions to optimize levels of nutrients that have beneficial effects within specific ranges are provided. Dietary plans, and formulations comprising dietary products that comprise optimized levels of nutrients derived from phytochemicals, antioxidants, vitamins, minerals, lipids, proteins, carbohydrates, probiotics, prebiotics, microorganisms and fiber. Diet plans and modular nutritional packages comprising food and drink items tailored according to consumer patterns typed by diet, age, size, gender, medical conditions, family history, climate and the like are provided.

### CROSS-REFERENCE TO RELATED APPLICATIONS

This patent application claims priority of U.S. Provisional Patent Application Ser. No. 61/393,235, filed Oct. 14, 2010 and U.S. Provisional Patent Application Ser. No. 61/415,096, filed Nov. 18, 2010. The contents of these patent applications are incorporated herein in their entirety by reference.

### TECHNICAL FIELD OF THE INVENTION

This invention relates to the field of nutritional compositions and formulations. In particular, the application relates to methods of selection of nutritional plans tailored to optimize benefits derived from nutrients. More particularly, the invention relates to formulations and dietary products that provide compositions comprising optimized levels of nutrients such as phytochemicals, antioxidants, vitamins, minerals, lipids, proteins, carbohydrates, probiotics, prebiotics, microorganisms and fiber.

### BACKGROUND OF THE INVENTION

The requirements of phytochemicals, lipids, and some other nutrients for human health are rather sensitive. There are many nutrient interactions and their range of healthful effectiveness is narrow and changes with diet type and/or demographic factors.

Formulations comprising lipids, antioxidants, phytochemicals, vitamins, minerals, microorganisms or a combination thereof, are traditionally provided as supplements or randomly added to nutritional or topical formulations. The focus is often on suppressing oxidation or inflammation, which ignores the fact that both oxidation and inflammation have a necessary role in physiology. Further, selective, repetitive, and excessive suppression may lead to dysregulation of inflammation with greater health consequences. Therefore, current approaches have the dangers of mismanaged and/or excessive delivery, which may be harmful particularly in combination with natural “nutrient rich foods,” including foods such as nuts, seeds, oils, grains, legumes, fruits, vegetables, seafood, herbs, and spices, packed with certain lipids, antioxidants, phytochemicals, vitamins, minerals, and microorganisms. Similarly, functional foods that are enriched with sterols, stanols, calcium, vitamin E, folic acid, omega-3, flavonoids, etc. can also be harmful out of context. The current approach leads to imbalanced or excessive consumption of these nutrients. As a result the prevalent approaches do not alleviate the disease burden.

To date there are no methods for matching naturally occurring foods such as nuts, seeds, oils, grains, legumes, fruits, vegetables, seafood, herbs, and spices to achieve optimal results. Instead focus is on additives, often to counter excesses. Currently there are no methods for creating delivery system(s) designed to deliver nutrients in an optimal range, such that a consumer can reach for products within the system, knowing that cumulative nutrients in the delivery system will keep them in a safe range. There is a need for the development of such system(s).

Therefore, it is desirable to develop a tailored nutritional program(s) or delivery system(s) where consumers are guided to consume naturally-occurring foods that have been matched keeping interactions, amounts, and consumer preferences in perspective. Further, the program(s) need to caution consumers against food types and amounts that may disrupt the nutritional optimization provided by the program. Within the broad parameters of personalization and moderate compliance, consumers may be at a reduced risk for chronic diseases, and with narrower parameters in personalization and greater compliance, greater health benefits may be achieved. To date tailored programs have been difficult to devise, particularly with regards to phytochemicals and lipid interactions and amounts.

The programs may be component or module based to allow flexibility and convenience for consumers. The benefits may be incremental with greater adherence to selection of components within the program. For example, lipid types and amounts are critical to health and can vary due to a number of factors, thus making the calibration complex for consumers to manage every day. Both the composition and the amounts need to be managed. For example, lipid requirement can be as much as 80 grams or 720 calories more for one family member (a 25-year-old male) than another (a 3-year-old child). This is further complicated because lipids do not mix homogenously with food; as such, individual portions may contain a disproportionate amount of lipids. Consequently, when lipids are supplemented within a given food preparation, an individual member may consume too little or too much of the lipids. Similarly, men may have a greater need for a nutrient than women. A tailored dietary component system may provide an effective solution.

As such there is a need for component based nutritional formulations, tailored diets and diet plans that provide optimized levels of nutrients such as phytochemicals, antioxidants, vitamins, minerals, lipids, proteins, carbohydrates, probiotics, prebiotics, microorganisms and fiber. Some of these nutrients are rarely the focus of diet plans, e.g. phytochemicals, yet too much or too little of such micronutrient can turn an otherwise beneficial micronutrient in the diet to have adverse effects.

### SUMMARY OF THE INVENTION

This invention relates to novel strategies for developing component based dietary formulations, and programs. In particular, the invention relates to generating tailored diets for consumers, wherein the nutrient levels are balanced to provide optimal benefits.

In certain aspects, the invention categorizes individuals into diet cohorts, for example, based on high meat, high plant, and high seafood diets. Consumers generally have a specific preference for the main foods such as red meat, seafood, or plant food. For example, vegetarians typically consume more vegetables, grains, and legumes, as compared to high-meat or high-seafood consumers. These dietary habits can help establish basic nutrients around which effective diet programs may be developed. Instead of randomly adding nutrients to a diet, there is a need to identify a series of diet types, e.g. plant, meat, or seafood heavy, and a series of consumer patterns typed by diet, age, size, gender, medical conditions, family history, climate and the like and then tailor nutritional compositions tailored to each series.

Therefore, in one aspect, the invention provides a method for customizing or selecting a nutritional formulation or plan for an individual, preferably a human. The invention in this aspect comprises determining for the individual, or categorizing the individual with respect to, a diet type (“cohort”). For example, the cohort may be high plant food, high meat (e.g., high red meat), or high seafood. In certain embodiments, the cohort is determined by the relative amounts of grains, vegetables, fruits, legumes, dairy, meats, seafood, herbs, sweeteners and beverages consumed by the individual, with a focus on foods rich in phytochemicals, and certain minerals and nutrients described herein, for which delivery should be controlled. The cohort may be determined based on an average daily consumption of such foods (weight, volume, or percent of calories). A nutritional program is then selected to balance certain lipids and nutrients by providing one or more nutritional formulations comprising natural oils, butters, margarines, nuts, seeds, herbs, vitamins, and minerals. These formulations deliver particular nutrients, such as lipids, phytochemicals, and minerals, to keep the individual in a safe range and thereby prevent or ameliorate the symptoms of chronic disease.

In certain embodiments, the nutritional formulation is packaged and marked for diet cohort, with a coding system for matching formulations to deliver the proper level of micronutrients, for the convenience of the individual in maintaining a balanced nutritional state. The formulations are marked to provide the frequency for consumption (e.g., three times daily, twice daily, or once daily, or a frequency of from one to five times per week). The individual's diet is balanced (by virtue of the nutritional formulation) with respect to lipids (C4:0, C22:6 omega 3, and others), carbohydrates, protein, vitamins, minerals, antioxidants, phytochemicals, prebiotics, probiotics, and fiber. In certain embodiments, the nutritional formulation is further customized based on the age, gender, size, climactic temperature, medical condition, or lipid tolerance of the individual. In some embodiments, the nutritional formulation is in the form of one or more of an oil blend, spread or dip, sauce or dressing, or small dessert, which may be for diurnal consumption in some embodiments.

In some embodiments, the diet is balanced by the delivery of one or more (e.g., from 2 to 10) nutritional formulations that collectively make up a nutritional program for an individual. The program may collectively meet the description of the nutritional plan of Tables 5, 6, 7, or 8. At least one formulation contains one or more of phytochemicals such as phytosterol or polyphenols non-limiting examples of which include, curcumin, coumarins, and rosemarinic acid. In these or other embodiments, the diet is also balanced by the nutritional formulation with respect to minerals such as selenium. That is, the individual's diet is characterized by the sufficiency of such nutrients, and customized nutritional formulations prepared to balance the individual's diet by delivering or withholding these nutrients and/or minerals. The formulation provides a balanced lipid profile for the individual leading to physiologically balanced levels of essential fatty acids, long chain polyunsaturated fatty acids (LCPUFA), saturated fatty acids, omega-3 fatty acids, including docosahexaenoic acid (DHA), arachidonic acid, linoleic acid, omega-6 fatty acid, and omega 6:omega-3 ratio. In these or other embodiments, the diet is balanced by the delivery or withholding of one or more of the following substances (or the oil thereof) in certain defined concentrations: peanuts, almonds, olives, soybeans, cashews, flaxseeds, pistachios, pumpkin seeds, sunflower seeds, sesame seeds, walnuts, anhydrous butter oil, and coconut meat. Other components for the nutritional formulations are disclosed herein.

In certain embodiments, the individual may exhibit signs or symptoms of a chronic medical condition selected from gout, diabetes (type 1 or type 2), heart disease, glycemia, insulinemia, metabolic syndrome, an age-related disease (e.g., macular degeneration), or an infectious disease, and such symptoms may be ameliorated by the balanced diet (via consumption of the nutritional formulation for a period of time). The nutritional formulations of the instant invention are suitable for prophylaxis or treatment of a medical condition or disease selected from menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases. Other features and/or components of the nutritional plan and nutritional formulations are described herein. Thus, in some embodiments, the individual is exhibiting signs and symptoms of such disease, and by consuming a customized nutritional formulation in accordance with the invention (tailored to the individual's diet cohort as described herein) for at least one week, two weeks, or one month, such symptoms are ameliorated. In some embodiments, medicaments are formulated based on a subject's dietary habits around typical consumption of phytochemicals, antioxidants, and other nutrients which may be administered with the diet plan. Appropriate supplements, medications or pharmaceutical drugs are administered to/by such dietary cohorts because their requirements, biochemistry, and gene expression may be influenced in a certain predictable way.

In another aspect, the invention provides nutritional compositions that may be modular/component systems of prepared or unprepared food, e.g. drinks, snacks, meals, desserts, cereals, salad, side dish, sauces, desserts, spreads etc, such that consumers can safely select a specific food or drink item, such as a bottle of juice, bar, a salad, a meal knowing that the nutrients derived from the components on the whole will keep them in a safe range. In certain embodiments, such components are packaged and marked for a particular cohort described herein, such that individuals can conveniently maintain nutritional balance without frequent nutritional counseling. The delivery may be in the form of novel dietary lipid programs comprising phytochemicals, antioxidants, vitamins, minerals, microorganisms and fiber designed for specific cohorts, comprising mutually complementing daily variety dosages of spread, oil blend, sauce, dressing, and dessert to fit the daily schedules, which could be convenient, appealing, and fun. Such programs minimize the possibilities and magnitude of adverse effects from inappropriate intake of nutrients, particularly phytochemicals and lipids and interactions among them.

Fine-tuning the dietary programs can be achieved by further tailoring for age, size, gender, medical conditions, lipid tolerance, family history, and climactic temperature, and the like. In some aspects, such tailored programs are developed utilizing computer modeling, which may be provided to the consumer through a user-friendly software or web interface, allowing the consumer to: identify their diet cohort (cohort's being described herein); select and/or design customized nutritional programs delivering optimal amounts of phytochemicals, minerals, and lipids, among others; and purchase/order the individualized nutritional compositions that make up the diet plan.

In one aspect, packages and kits of prepared or unprepared food are provided to support specific aspects of the nutritional plan. In some embodiments, the packages and kits comprise component or modular systems comprising vegetable or vegetable juice packs, fruit or fruit juice packs, dry grain packs, cereal packs, legume/grain/nuts and/or seed packs, meat/seafood packs, herbs, lipids, desserts, milks, yogurts and the like, or a combination thereof. In some embodiments, the kits comprise from 2 to 20, or from 5 to 10 nutritional formulations, which collectively, balance the individual's diet within the parameters disclosed in one of Tables 5 to 8. The nutritional formulations may be designed to, collectively, comprise at least 40%, at least 50%, at least 60%, or at least 80% of the individual's caloric intake. In some embodiments, the kits and packages comprise food suitable for consumption by babies and include, but are not limited to soybean-based formula, milk formula, standard milk formula, follow-on milk formula, toddler milk formula, hypoallergenic milk formula, prepared baby food, dried baby food and other baby food.

In one aspect, food items recommended in a diet plan or contained in a specific component or module are selected based on the methods of cooking, processing or manufacturing used in preparing the food items such that optimal nutrient content is achieved, and/or desired activation or inactivation of nutrients particularly phytochemicals is achieved.

Further aspects and embodiments of the invention will be apparent from the following detailed description of the invention.

### DETAILED DESCRIPTION OF THE INVENTION

Universal supplementation of monounsaturated, omega-6, omega-3, other fatty acids, antioxidants, phytochemicals, vitamins, or minerals, and microorganisms without regard to the context has not been effective. Sensitive requirements are materially altered by a number of nutritional and demographic factors. Further, while many nutritional systems focus on the protein and/or carbohydrate component of the diet, proteins and carbohydrates affect health patterns mostly when consumed in large amounts, e.g. a gram or more. On the other hand, microgram amounts of nutrients such as some lipids, antioxidants, phytochemicals, vitamins, minerals, probiotics, prebiotics, and microorganisms, can have significant effect on health. Thus, an object of the present invention is to balance nutrition based on supplementation with lipids, antioxidants, phytochemicals, vitamins, minerals, probiotics, prebiotics, and/or microorganisms.

The present invention relates, in-part, to the surprising finding that, while phytochemicals, lipids, antioxidants, vitamins, minerals, and microorganisms have a narrow window of healthful effects, and that the requirements change based on the complement of nutrients, individualized diet plans can nevertheless be designed with surprising simplicity and accuracy. Therefore, the invention provides methods for preparing nutritional plans, and provides nutritional formulations (including complementing nutritional formulations), such that total consumption of these key nutrients is kept in a safe range. Further benefit can be derived by tailoring them to diet cohort defined at least in part by protein and carbohydrate consumption. Further benefit can be derived by tailoring these formulations to diet cohort defined at least in part by demographic factors including one or more of: age, gender, size, medical condition, family history, and climate. Such methods would lead to reduced risk for chronic diseases, and achieve greater health benefits.

The following description of example embodiments is, not to be taken in a limited sense. The scope of the present invention is defined by the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. Nothing herein is to be construed as an admission concerning the content of the prior art, that the invention is not entitled to antedate any particular disclosure by virtue of prior invention.

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention.

### DEFINITIONS

As used herein, the term “phytochemical” refers to any natural molecule of plant origin. They are found in fruits, vegetables, beans, grains, and other plants. The terms “phytochemical” and “phytonutrient” are used interchangeably to describe the active components of plants. Commonly known phytonutrients or phytochemicals include (but are not limited to) antioxidants, flavonoids, flavones, isoflavones, catechins, anthocyanidins, isothiocyanates, carotenoids, allyl sulfides, polyphenols, terpenes, limonoids, lipids, phytosterols, beta carotene, ascorbic acid (vitamin C), folic acid, and vitamin E. Phytochemicals that the nutritional plan may control, and exemplary sources, are listed in Table 1. These phytochemicals/sources are controlled in the construction of the diet plan, and their delivery substantially controlled by virtue of a one, two, or three complementing formulations of natural oils, butters, margarines, nuts, seeds, herbs, vitamins, and minerals. Optionally, these formulations may take the form of a conventional supplement, such as a capsule for oral administration, or alternatively a topical formulation.

As used herein, the term “lipid” refers to any fat-soluble (lipophilic) molecule. These include (but are not limited to) components of vegetable oils, components of seed oils, triglycerides, waxes of triglycerides, and phospholipids. As used herein, the term “lipid” comprises a source of lipids or fats comprising any suitable lipid or lipid mixture. For example, the lipid source may include, but is not limited to, vegetable fat (such as olive oil, peanut oil, corn oil, sunflower oil, rapeseed oil, soy oil, palm oil, coconut oil, canola oil, lecithins, walnuts, flaxseeds, and the like) and animal fats (such as milk fat), structured lipids or other modified lipids such as medium chain triglycerides. As used in the nutritional formulations disclosed herein, the lipid is a component of a dietary food item and/or added individually as a supplement.

In some embodiments, the compositions of the present disclosure include one or more of the following fatty acids: Saturated fatty acids: butyric (C4:0), lauric (C12:0), myristic (C14:0), palmitic (C16:0), stearic (C18:0), and arachidic (20:0); monounsaturated fatty acids: myristoleic (C14:1), palmitoleic (C16:1); omega-9 fatty acids: oleic (C18:1), gadoleic (C20:1), erucic (C22:1), and nervonic (C24:1); omega-6 fatty acids: linoleic (C18:2), conjugated-linoleic (C18:2), gamma-linolenic (C18:3), eicosadienoic (C20:2), di-homo-gamma-linolenic (C20:3), and arachidonic (C20:4); and omega-3 fatty acids: alpha-linolenic (C18:3), stearidonic (C18:4), eicosapentaenoic (C20:5), docosapentaenoic (C22:5), and docosahexaenoic (C22:6) fatty acids.

As used herein, a “prebiotic” is a food substance that selectively promotes the growth of beneficial bacteria or inhibits the growth or mucosal adhesion of pathogenic bacteria in the intestines. The prebiotic can be acacia gum, alpha glucan, arabinogalactans, arabinoxylans, beta glucan, dextrans, fructooligosaccharides, galactooligosaccharides, galactomannans, gentiooligosaccharides, glucooligosaccharides, guar gum, inulin, isomaltooligosaccharides, lactosucrose, lactulose, levan, maltodextrins, partially hydrolyzed guar gum, pecticoligosaccharides, resistant starches, retrograded starch, soy oligosaccharides, sugar alcohols, xylooligosaccharides, or their hydrolysates, or combinations thereof. For example, prebiotics are defined by Glenn R. Gibson and Marcel B. Roberfroid, “Dietary Modulation of the Human Colonic Microbiota: Introducing the Concept of Prebiotics,” J. Nutr. 1995 125: 1401-1412. Prebiotics are fermented by the gastrointestinal microflora and/or by probiotics.

As used herein, probiotic micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al. Trends Food Sci. Technol. 1999: 10 107-10. The probiotic can be of bacterial, yeast, or fungal origin, including Saccharomyces, Debaromyces, Candida, Pichia, Torulopsis, Aspergillus, Rhizopus, Mucor, Penicillium, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

As used herein, the term “protein” comprises a protein or polypeptide obtained from a source selected from dietary protein including, but not limited to animal protein (such as milk protein, meat protein or egg protein), vegetable protein (such as soy protein, wheat protein, rice protein, canola and pea protein), or a combination thereof. In another embodiment, the compositions or formulations include one or more amino acids selected from: Isoleucine, Alanine, Leucine, Asparagine, Lysine, Aspartate, Methionine, Cysteine, Cystine, Phenylalanine, Glutamate, Threonine, Glutamine, Tryptophan, Citrulline, Glycine, Valine, Proline, Serine, Tyrosine, Arginine, Histidine, or a combination thereof.

As used herein, the term “carbohydrate” refers to a source of carbohydrates comprising any suitable carbohydrate, including, but not limited to, sucrose, lactose, glucose, fructose, corn syrup solids, maltodextrin, modified starch, amylose starch, tapioca starch, corn starch, isomalt, isomaltulose, or combinations thereof. As used in the nutritional formulations disclosed herein, the carbohydrate is a component of a dietary food item and/or added individually as a supplement.

As disclosed herein, the nutritional composition includes minerals, or supplements containing such minerals, in a form that promotes metabolic alkalinity versus acidity. The minerals are provided attached to various organic acids, amino or fatty acids, or naturally occurring as part of a real food. For example, different forms of magnesium, calcium or aluminum are suitable for affecting acid-base balance.

The compositions/formulations disclosed herein can be included in a nutritional or nutraceutical composition together with additional active agents, carriers, vehicles, excipients, or auxiliary agents identifiable by a person skilled in the art upon reading of the present disclosure.

Subject as used herein refers to humans and non-human primates and any other organisms which can benefit from the agents of the present disclosure. There is no limitation on the type of animal that could benefit from the presently described agents. A subject regardless of whether it is a human or non-human organism may be referred to as a patient, individual, animal, host, or recipient. In certain preferred embodiments, the subject is a human.

### Abbreviations

The following abbreviations are used throughout the application: AA, arachidonic acid (20:4n-6); ADHD, attention deficit hyperactivity disorder; ALA, alpha-linolenic acid (18:3n-3); γT, alpha-tocopherol; COX, cyclooxygenase; DSD, delta-5-desaturase; D6D, delta-6-desaturase; DGLA, dihomo-gamma-linolenic acid (20:3n-6); DHA, docosahexaenoic acid (22:6n-3); HNF, hepatic nuclear factor; EFA, essential fatty acids; EPA, eicosapentaenoic acid (20:5n-3); GLA, gamma-linolenic acid (18:3n-6); GSHpx, glutathione peroxidase; γT, gamma-tocopherol; IL, interleukin; LA, linoleic acid (18:2n-6); LCPUFA, long-chain PUFA (DGLA, AA, EPA, and DHA); LPO, lipid peroxidation products; LT, leukotriene; LXR, liver X receptor; MUFA, monounsaturated fatty acids; NFkB, nuclear factor kB; OA, oleic acid (18:1n-9); PG, prostaglandin; PPAR, peroxisome proliferator activated receptor; PUFA, polyunsaturated fatty acid; SCD, stearoyl CoA desaturase also known as delta-9-desaturase; Se-GSHpx, Se-dependent glutathione peroxidase; SFA, saturated fatty acids; SOD, superoxide dismutase; SREBP, sterol regulatory element-binding proteins; TNF, tumor necrosis factor; TX, thromboxane; UCP, uncoupling proteins.

The invention disclosed herein relates to development of nutritional compositions and/or formulations tailored to individual preferences that balance phytochemicals, antioxidants, vitamins, minerals, acid-base, lipids, proteins, carbohydrates, probiotics, prebiotics, microorganisms, fiber, and the like. Nutritional plans are based primarily on consumption of food from preferred natural sources. Levels and types of nutrients in each food item are considered in developing a nutritional plan keeping interactions in perspective that provides nutrients at levels that have exemplary health benefits. Nutritional plans are tailored to fit the primary dietary preferences of consumers.

### Nutritional Plans and Influencing Factors

In one aspect, the invention provides a method for customizing or selecting a nutritional plan for an individual. The nutritional plan comprises from 2 to about 20 (or from 2 to about 10) nutritional formulations, which are mutually complementing to balance certain micronutrients described herein. In certain embodiments, the nutritional plan comprises from 4 to about 12 or from 4 to about 10 mutually complementing formulations (e.g., complementing with respect to micronutrients). In certain embodiments, one, two, or three of these formulations deliver (collectively) at least 50%, or at least 75%, or at least 90% of the set of micronutrients, with the remaining formulations balanced with respect to basic dietary considerations, such as protein intake, carbohydrate intake, and/or caloric intake, for example. Lipid intake is also balanced, but in-part the balance is achieved by the delivery of the micronutrient formulation. For the one, two, or three formulations comprising the substantial level of micronutrients, a subset of from 3 to about 10 formulations can be prepared for selection between individuals, thereby allowing for cost-effective individualization. For example, in a particular example, the formulations delivering the micronutrients may deliver polyphenols at about 5, 10, 15, 20, 45, 70, 95, 115, 140, or 165 mg/day; and (respectively) folate at about 100, 200, 300, 400, 500, 600, 700, 800, 900, or 1000 mcg/day; phytosterols (at respectively) about 150, 200, 250, 300, 350, 450, 550, 650, 750, or 850 mg/day; and Se at about 5, 10, 15, 20, 35, 55, 75, 95, 115, or 135 mcg/day (respectively). In some embodiments, these values may vary by up to 10% or 20%.

The invention in this aspect comprises determining for the individual, or categorizing the individual with respect to, a diet type or “cohort.” For example, the diet type may be high plant food, high meat (e.g., high red meat), or high seafood. In certain embodiments, the diet type is determined by the relative amounts of grains, vegetables, fruits, legumes, dairy, meats, seafood, herbs, sweeteners and beverages consumed by the individual. A nutritional program is then selected to balance certain phytochemicals including lipids and other nutrients by the delivery of one or more nutritional formulations comprising one or more of natural oils, butters, margarines, nuts, seeds, herbs, vitamins, and minerals. For example, the nutritional formulation may be packaged and marked for diet type or cohort, for the convenience of the individual. In certain embodiments, the packaging of the nutritional formulations may comprise components or modules each comprising all or part of a dietary cohort's nutritional requirements. In certain embodiments, the nutritional formulation is further customized based on the age, gender, climactic temperature, medical condition, or lipid tolerance of the individual. Balancing diet plans based on certain demographics is described in WO 2009/131939, which is hereby incorporated by reference.

For example, diet plans and nutritional kits can be prepared as follows.

Dietary components (such as those described herein) are grouped as: legume, grain, vegetable, fruit, meat, seafood, herb, spice, nut, seeds, oil, or butter.

Food items from the list are selected that have a significant level of sensitive nutrients (see, e.g., Table 1), such as polyphenols, phytosterols, fat soluble vitamins/substances A, D, E, K, lipids, folate, and Se. These should be controlled. Food sources having significant levels of these micronutrients are described herein, and are known in the art. Where a layer or part of the sensitive food item can be removed (e.g. bran, husk, germ, or skin) to remove the significant levels of micronutrient, then the part is removed and the food item is regrouped with its basic category (e.g., legume or grain). In some embodiments, a method of processing as described later is used to arrive at optimal nutrient content or activity.

With like items grouped together, a combination of grains is created, as described herein and as shown in one of Tables 5-8 for example. In certain embodiments, grains with strong properties e.g. barley, spelt, quinoa, millet, spelt oats, and rye are controlled. For example, collectively, in certain formulations, these components may make up less than 70%, less than 50%, less than 40%, less than 30%, or less than 20% of carb calories. These steps are repeated for legumes, vegetables, and fruits. The amounts of soy, pink lentils, black beans, and pigeon peas are also controlled, since these items are high in flavonoids. In various embodiments, these items make up less than 70%, less than 50%, less than 40%, less than 30%, or less than 20% of protein calories.

For meat and seafood items, these comprise less than 70%, less than 50%, less than 40%, less than 30%, or less than 20% of protein calories.

The remaining nutrients needed to balance the nutritional plan are supplied by one, two, or three or more formulations comprising herbs, spices, nuts, seeds, oils, butters, and sweeteners, which are described in detail herein. Thus, the entire nutritional plan in some embodiments meets the description in one of Tables 5 to 8. For example, for this micronutrient formulation, grains, legumes, vegetables, fruits, herbs, seeds, or a combination thereof; in whole, stripped-down, or processed form; are prepared to arrive at a healthful dosage of phytochemicals (polyphenols, sterols, coumarins, isoflavones (Daidzein, Genistein, Glycitein), flavonoids, bran, endosperm etc.).

The set of formulations can be fine-tuned by cohorts, such as for heavy meat, vegetarian, and heavy seafood. For each cohort, it is important to identify which phytochemicals, minerals, and/or nutrients are likely to be over- or underconsumed. For example, for the meat cohort: it is likely that consumption of phytosterols, polyphenols, and isoflavones is inadequate, and thus should be supplemented accordingly. If most protein calories are met by meat, then herbs, nuts and seeds can replace added fats. If there is room for additional protein calories, then legumes (black beans, kidney beans, peas, soy, pigeon peas, black gram, chickpeas) can be used. For heavy seafood diets, it will likely be necessary to avoid nuts, seeds, and certain whole grains. For vegetarians, the nutritional plan must guard against excess of phytochemicals particularly phytosterols, polyphenols, isoflavones, and make up for potential protein deficiency. The actual level for each phytochemical, mineral, or nutrient consumed too much or too little in each cohort, can be computed.

By classifying an individual as meat, plant, or seafood-base cohort, the following factors can be taken into account. In certain embodiments, cohorts are specifically defined by the amount or presence of the following factors.

In certain embodiments, the nutritional formulation is balanced, with respect to the individual, for essential fatty acids and their metabolites, long-chain polyunsaturated fatty acids (LCPUFA), eicosanoids, monounsaturated fatty acids, and saturated fatty acids, through the delivery of lipids, phytochemicals, nutrients, minerals, and other components.

In some embodiments, the individual's diet cohort is defined, at least in-part, by consumption of and requirement for essential fatty acids, and which may be implemented by the defining cohorts as plant-based, meat-based, or seafood-based, in some embodiments. For example, essential fatty acids (EFA) and their metabolites, long-chain polyunsaturated fatty acids (LCPUFA) and various eicosanoids play an important role in human health. Monounsaturated and saturated fatty acids also have a significant role in health. However, the latter can inhibit the activity and bioavailability of EFA and LCPUFA. Genders differ in their ability to metabolize lipids due to sex hormones and differential gene expression. Change in hormone status may also change lipid requirements with age. Further, of the macronutrients, lipids are the most susceptible to oxidative stress, which is one of the most likely causes of aging. Synergistic and managed use of different antioxidants is of benefit to human health. Sudden and wide fluctuations in fatty acids consumption can alter the immune response, which is dose-dependent, the excitability of neural and muscle cells and neurotransmission, and androgen production. Thus sudden and large alterations in fatty acids consumption may cause compromised immunity and physiological disturbances.

In some embodiments, the diet cohort is defined, at least in-part, by the individual's consumption of and requirement for omega-6, omega-3, and omega-9 fatty acids, including one or more of the omega-6:omega-3 ratio, the omega-9:omega-6 ratio, ratio of monounsaturated fatty acids to polyunsaturated fatty acids, and ratio of monounsaturated fatty acids to saturated fatty acids.

In these and other embodiments, the individual's diet cohort is defined at least in-part by the individual's consumption of antioxidants, phytochemicals, vitamins, and minerals, including particular antioxidants, phytochemicals, vitamins, and minerals described herein. In some of these embodiments, the cohort is further defined by gender, age, size, and climactic temperature for the individual, which will affect the individual's requirement for such nutrients. A number of factors can influence metabolism, including antioxidants, phytochemicals, vitamins, minerals, hormones, and microorganisms as well as the gender, genetics and age of the individual, and climactic temperature.

Nutritional programs are developed based on the observation that phytochemicals, antioxidants, vitamins and minerals, microorganisms significantly alter the sensitivity of lipid requirement and metabolism. Thus, the diet cohort may be defined in some embodiments by the requirement for omega-6, omega-3, and omega-9 fatty acids, including one or more of the omega-6:omega-3 ratio, the omega-9:omega-6 ratio, ratio of monounsaturated fatty acids to polyunsaturated fatty acids, and ratio of monounsaturated fatty acids to saturated fatty acids; and this requirement used to supplement or withdraw one or more of phytochemicals, antioxidants, vitamins, minerals, and microorganisms from the individual's diet, using a customized nutritional composition.

While a number of factors can influence fatty acid metabolism such as the presence of other fatty acids, antioxidants, phytochemicals, vitamins, minerals, hormones, and microorganisms as well as the gender, genetics and age of the individual consumer, and climactic temperature, the present invention provides a simple yet accurate method for determining an individual's requirement for fatty acids, and a convenient and effective nutritional supplementation program. While in certain embodiments the individual's requirements are determined by identifying a basic diet cohort (e.g., meat, plant, or seafood, and optionally one or more of gender, size, age, and climactic temperature), additional influencing factors may optionally be considered in defining the diet cohort, and these influencing factors are described below.

### Desaturase Modulators

In certain embodiments, the individual's diet cohort is defined, at least in-part, by the individual's consumption of, and requirement for, desaturase modulators. In these embodiments, the individual is provided a nutritional supplement and/or program to balance the requirement. Desaturase modulators include essential fatty acids, vitamin A, curcumin, sesamin, and phytosterols.

The desaturases D6D and D5D are involved in the production of potent LCPUFA. Several nutritional, hormonal, and genetic factors can influence the activity of the desaturases. In response to increase or decrease of EFA levels, the desaturases may rapidly change in activity levels. Thus, a large and sudden increase in omega-6 fatty acids from deficient conditions may lead to sudden surge of LCPUFA, its metabolites, and inflammation. The limited desaturase activity in certain pathological states might be due to or exacerbated by other endogenous or exogenous factors rather than an enzymatic defect.

Males and females differ in their ability to synthesize long-chain omega-3 fatty acids from ALA as hormones play a role. Estradiol may increase, whereas testosterone may decrease the production of LCPUFA from LA and ALA. Omega-3 pathway is more responsive to hormonal treatment than omega-6 pathway. In females, the conversion from ALA to DHA may be as high as 9%, whereas for males it may be 0.5-4% resulting in higher DHA concentration in plasma lipids, without significant differences in their consumption of protein, carbohydrate, total fat, alcohol, individual fatty acids and selected nutrients. Growth hormones have been found to increase the D6D activity and LCPUFA in animal models. Vitamin A has been shown to down regulate the expression of D5D. In addition, some phytochemicals, particularly curcumin and sesamin, have also been shown to influence D5D function. D5-desaturation of omega-6 fatty acids was down regulated, whereas D5-desaturation omega-3 fatty acids was up regulated. Fujiyama-Fujiwara Y, et al. Effects of sesamin and curcumin on delta 5-desaturation and chain elongation of polyunsaturated fatty acid metabolism in primary cultured rat hepatocytes. J Nutr Sci Vitaminol (Tokyo). August 1992; 38(4):353-363. 1995; 41(2), 217-225. Phytosterols have been shown to increase the activity of D6D, D5D, and SCD.

The most potent regulator of desaturase activity is the cellular LCPUFA availability. Under normal physiological conditions cellular LCPUFA is maintained in a narrow range by regulation of desaturase transcription.

### Phytochemicals

In certain embodiments, the individual's diet cohort is defined, at least in-part, by the individual's consumption of, and requirement for, phytochemicals. In these embodiments, the individual is provided a nutritional supplement and/or program to balance the requirement. Phytochemicals in certain embodiments are one or more of those in Table 2. In certain embodiments, the cohort is defined by the approximate level of consumption of the sources of such phytochemicals listed in Table 2.

A key ingredient in optimizing dietary programs comprises providing the proper types and amounts of phytochemicals in the nutritional plan. Phytochemicals (phytoalexins, plant matter, natural molecules contained in plants) have powerful properties, but healthful effects are available within narrow ranges of amounts included in the diet particularly because they have cumulative effects.

In general, phytochemicals: (a) have antioxidant properties, change oxidation of lipids and other molecules; (b) may turn into prooxidants at high amounts or due to some interactions; (c) modulate gene expression; stimulate synthesis of adaptive proteins/genes for cytoprotective, detoxifying and antioxidant enzymes; (d) maintain genome integrity; (e) modulate cell signaling pathways and membrane, cytoplasmic, and nuclear enzymatic reactions; (f) dampen cellular hyperproliferation and hyperactivity, promote apoptosis of genetically unstable cells; (g) accumulate in cell membranes causing alterations of cell shape and modulation of the bilayer material properties (bilayer thickness, fluidity and elasticity) that affect membrane proteins and ion channels; (h) inhibit inflammation, e.g. transcription of NFkB, which regulates broad range of cytokine genes involved in inflammation (e.g. sulforaphane, curcumin, zerumbone); or activate PPAR-gamma, which may modulate anti-inflammatory genes and inhibit NFkB (e.g. curcumin, capsaicin, ginsenosides, hesperidin, and resveratrol); (i) may excessively suppress oxidation and/or certain inflammatory molecules or pathways; body may then upregulate compensatory mechanisms; (j) may inhibit mitochondrial function; (k) may lead to acidosis particularly when consumed with omega-3 and unbalanced or inadequate lipids (xanthones have been shown to cause acidosis, there are quite likely other phytochemicals that cause acidosis); (1) may alter metabolism and activity of lipids and their metabolites; (m) may increase the requirement for Omega-6, and some other fatty acids; and (n) may reduce the requirement or tolerance for omega-3 (e.g. certain polyphenols enhance synthesis of long-chain omega-3 from its precursor, but may impede formation of long-chain omega-6).

TABLE 1
List of common/known phytochemicals and plant matter and their exemplary sources.

MONOPHENOLS:
Apiole (parsley)
Carnosol (rosemary)
Carvacrol (oregano, thyme)
Dillapiole (dill)
Rosemarinol (rosemary)
POLYPHENOLS: (flavonoids, phenolic acids, lignans, stilbenes)
Flavonoids
Flavonols: Quercetin (onions, tea, wine, apples, cranberries, buckwheat, beans),
Gingerol (ginger), Kaempferol (strawberries, gooseberries, cranberries, peas,
brassicates, chives), Myricetin (grapes, walnuts), Rutin (citrus fruits, buckwheat,
parsley, tomato, apricot, rhubarb, tea). Isorhamnetin, Proanthocyanidins
procyanidins, prodelphinidins and propelargonidins, apples, maritime pine bark,
cinnamon, aronia fruit, cocoa beans, grape seed, grape skin, red wine
Flavones: Chrysin, Apigenin (chamomile, celery, parsley) Luteolin, Tricetin, Disometin
etc Parsley, capsicum pepper
Flavanones: Naringenin (citrus), Hesperidin (citrus), Dihydroquercetin etc
Orange juice, grape fruit, lemon peel & juice etc, Eriodictyol.
Flavan3ols: Catechins (white tea, green tea, black tea, grapes, wine, apple juice,
cocoa, lentils, black-eyed peas), Silymarin, Silibinin, Taxifolin, (+)-Catechin,
(+)-Gallocatechin, (−)-Epicatechin, (−)-Epigallocatechin, (−)-Epigallocatechin
gallate (EGCG) - green tea;
(−)-Epicatechin 3-gallate (ECG), Theaflavin - black tea; Theaflavin-3-gallate -
black tea; Theaflavin-3′-gallate - black tea; Theaflavin-3,3′-digallate - black tea;
Thearubigins etc Cocoa, chocolates, cocoa beverages, beans, cherry, grapes, red wine,
cider, blackberry etc
Isoflavones: Daidzein (formononetin) - soy, alfalfa sprouts, red clover,
chickpeas, peanuts, other legumes. Genistein (biochanin A) - soy, alfalfa
sprouts, red clover, chickpeas, peanuts, other legumes. Glycitein - soy.
Chalcones:
Anthocyanins and Anthocyanidins: Pelargonidin - bilberry, raspberry,
strawberry. Peonidin - bilberry, blueberry, cherry, cranberry, peach. Cyanidin -
red apple & pear, bilberry, blackberry, blueberry, cherry, cranberry, peach, plum,
hawthorn, loganberry, cocoa. Delphinidin - bilberry, blueberry, eggplant.
Malvidin - bilberry, blueberry. Petunidin
Dihydroflavonols
Chalconoids
Coumestans (phytoestrogens) Coumestrol - red clover, alfalfa sprouts, soy, peas,
brussels sprouts.
phloretin.
Phenolic acids
Ellagic acid - walnuts, strawberries, cranberries, blackberries, guava, grapes.
Gallic acid - tea, mango, strawberries, rhubarb, soy.
Salicylic acid - peppermint, licorice, peanut, wheat.
Tannic acid - nettles, tea, berries.
Vanillin - vanilla beans, cloves.
Capsaicin - chilli peppers.
Curcumin - turmeric, mustard. (Oxidizes to vanillin.)
Lignans (phytoestrogens) - seeds (flax, sesame, pumpkin, sunflower, poppy), whole
grains (rye, oats, barley), bran (wheat, oat, rye), fruits (particularly berries) and vegetables.
Silymarin - artichokes, milk thistle.
Matairesinol - flax seed, sesame seed, rye bran and meal, oat bran, poppy seed,
strawberries, blackcurrants, broccoli.
Secoisolariciresinol - flax seeds, sunflower seeds, sesame seeds, pumpkin,
strawberries, blueberries, cranberries, zucchini, blackcurrant, carrots.
Pinoresinol and lariciresinol - sesame seed, Brassica vegetables
enterolactone, enterodiol
Stilbenes
Resveratrol - grape skins and seeds, wine, nuts, peanuts, berries
Pterostilbene - grapes, blueberries
Piceatannol - grapes
Punicalagins - pomegranates
Hydroxycinnamic acids
Caffeic acid - burdock, hawthorn, artichoke, pear, basil, thyme, oregano, apple,
rosemary, coffee
Chlorogenic acid - echinacea, strawberries, pineapple, coffee, sunflower,
blueberries.
Cinnamic acid - cinnamon, aloe.
Ferulic acid - oats, rice, artichoke, orange, pineapple, apple, peanut.
Coumarin - citrus fruits, maize.
Tyrosol esters
Tyrosol - olive oil
Hydroxytyrosol - olive oil
Oleocanthal - olive oil
Oleuropein - olive oil
TERPENES (ISOPRENOIDS)
Carotenoids (tetraterpenoids)
Carotenes - orange pigments
α-Carotene - to vitamin A, in carrots, pumpkins, maize, tangerine, orange.
β-Carotene - to vitamin A, in dark, leafy greens and red, orange and yellow fruits and
vegetables.
γ-Carotene
δ-Carotene
Lycopene - Vietnam Gac, tomatoes, grapefruit, watermelon, guava, apricots,
carrots, autumn olive.
Neurosporene
Phytofluene - star fruit, sweet potato, orange.
Phytoene - sweet potato, orange.
Xanthophylls - yellow pigments.
Canthaxanthin - paprika.
Cryptoxanthin - mango, tangerine, orange, papaya, peaches, avocado, pea,
grapefruit, kiwi.
Zeaxanthin - wolfberry, spinach, kale, turnip greens, maize, eggs, red pepper,
pumpkin, oranges.
Astaxanthin - microalge, yeast, krill, shrimp, salmon, lobsters, and some crabs
Lutein - spinach, turnip greens, romaine lettuce, eggs, red pepper, pumpkin,
mango, papaya, oranges, kiwi, peaches, squash, legumes, brassicates, prunes,
sweet potatoes, honeydew melon, rhubarb, plum, avocado, pear.
Rubixanthin - rose hips.
Monoterpenes
Limonene - oils of citrus, cherries, spearmint, dill, garlic, celery, maize,
rosemary, ginger, basil.
Perillyl alcohol - citrus oils, caraway, mints.
Saponins - soybeans, beans, other legumes, maize, alfalfa.
Lipids
Phytosterols - almonds, cashews, peanuts, sesame seeds, sunflower seeds, whole
wheat, maize, soybeans, many vegetable oils.
Campesterol - buckwheat.
beta Sitosterol - avocados, rice bran, wheat germ, corn oils, fennel, peanuts,
soybeans, hawthorn, basil, buckwheat.
gamma sitosterol
Stigmasterol - buckwheat.
Tocopherols (vitamin E)
omega-3,6,9 fatty acids - dark-green leafy vegetables, grains, legumes, nuts.
gamma-linolenic acid - evening primrose, borage, blackcurrant.
Triterpenoid
Oleanolic acid - American pokeweed, honey mesquite, garlic, java apple, cloves,
and many other Syzygium species.
Ursolic acid - apples, basil, bilberries, cranberries, elder flower, peppermint,
lavender, oregano, thyme, hawthorn, prunes.
Betulinic acid - Ber tree, white birch, tropical carnivorous plants Triphyophyllum
peltatum and Ancistrocladus heyneanus, Diospyros leucomelas a member of the
persimmon family, Tetracera boiviniana, the jambul (Syzygium formosanum), and
many other Syzygium species.
Moronic acid - Rhus javanica (a sumac), mistletoe
BETALAINS
Betacyanins
betanin - beets, chard
isobetanin - beets, chard
probetanin - beets, chard
neobetanin - beets, chard
Betaxanthins (non glycosidic versions)
Indicaxanthin - beets, sicilian prickly pear
Vulgaxanthin - beets
ORGANOSULFIDES
Dithiolthiones (isothiocyanates)
Sulphoraphane - Brassicates.
Thiosulphonates (allium compounds)
Allyl methyl trisulfide - garlic, onions, leeks, chives, shallots.
Diallyl sulfide - garlic, onions, leeks, chives, shallots.
INDOLES, GLUCOSINOLATES/SULFUR COMPOUNDS
Indole-3-carbinol - cabbage, kale, brussels sprouts, rutabaga, mustard greens,
broccoli.
sulforaphane - broccoli
3,3′-Diindolylmethane or DIM - broccoli family
Sinigrin - broccoli family
Allicin - garlic
Alliin - garlic
Allyl isothiocyanate - horseradish, mustard, wasabi
Piperine - black pepper
Syn-propanethial-S-oxide - cut onions.
PROTEIN INHIBITORS
Protease inhibitors - soy, seeds, legumes, potatoes, eggs, cereals.
OTHER ORGANIC ACIDS
Oxalic acid - orange, spinach, rhubarb, tea and coffee, banana, ginger, almond,
sweet potato, bell pepper.
Phytic acid (inositol hexaphosphate) - cereals, nuts, sesame seeds, soybeans,
wheat, pumpkin, beans, almonds.
Tartaric acid - apricots, apples, sunflower, avocado, grapes.
Anacardic acid - cashews, mangoes.

See Dr. Duke's Phytochemical and Ethnobotanical Databases (available on the web at ars-grin.gov/duke/) for details on phytochemicals in natural foods and their known or presumed activities.

### Lipids and Metabolites

In certain embodiments, the individual's diet cohort is defined, at least in-part, by the individual's consumption of, and requirement for, omega-3, omega-6, omega-9 fatty acids, and optionally, fat soluble vitamins including A, D, E, and K. In these embodiments, the individual is provided a nutritional supplement and/or program to balance the requirement. Optimal levels for basic dietary cohorts of meat, plant, and seafood heavy diets is disclosed herein (see Tables 6 to 8).

Lipids include a group of phytochemicals that include omega-3, -6, -9 fatty acids, other fatty acids, waxes, sterols, fat-soluble vitamins A, D, E and K. Phytosterols are a subgroup of lipids, more than 200 steroid compounds similar to cholesterol are found in plants.

A large part of the human sensitivity to lipids is due to the actions of essential fatty acids (EFA) and their metabolites. Eicosanoids, EFA metabolites, are involved in various physiological and pathological processes, including blood vessel constriction, dilation, blood pressure regulation, platelet aggregation, and modulation of inflammation. Generally, eicosanoids of AA origins produce a vigorous response, whereas eicosanoids of EPA origins produce a muted response. Additionally, AA, EPA, and DHA are precursors for lipoxins, resolvins, and neuroprotectins with anti-inflammatory properties. Though LCPUFA modulate a number of biological functions through eicosanoids, the fatty acids are highly active as components of cell membranes in pinocytosis, ion channel modulation, and gene regulation.

It is important to balance omega-6 and omega-3 fatty acids in human nutrition for optimal function of cellular membranes and for balance between eicosanoids produced from omega-6 and omega-3 fatty acids. The present consumption pattern, omega-6-to-omega-3 ratios of 15:1-17:1 in Western diets, has been cited as one of the dietary component significantly associated with modern chronic diseases. Simopoulos A P. Evolutionary aspects of diet, the omega-6/omega-3 ratio and genetic variation: nutritional implications for chronic diseases. Biomed Pharmacother. November 2006; 60(9):502-507.

In addition to the effects of ratios of fatty acids, plasma and/or serum lipids comprising high proportions of palmitic (16:0), palmitoleic (16:1), and DGLA, and a low proportion of LA and PUFA are associated with type-2 diabetes, myocardial infarction, stroke, left ventricular hypertrophy, and metabolic syndrome. High D6D and SCD (stearoyl CoA desaturase), and low D5D activity has been independently associated with cardiovascular disease risk markers, including insulin resistance and low-grade inflammation, and cardiovascular and total mortality. Altered endogenous desaturase levels might contribute to the mortality risks. Defect in D6D and D5D may be a factor in the initiation and progression of atherosclerosis and often associated diseases such as obesity, diabetes mellitus, and hypertension.

Omega-3 fatty acids of seafood origin include, but are not limited to, salmon, herring, mackerel, anchovies and sardines. Omega-3 fatty acids of botanical origin include, but are not limited to, chia, kiwifruit, perilla, flaxseed, lingonbeny, camelina, purslane, black raspberry, butternuts, hempseed, walnut, pecan nut, and hazel nut.

### Non-Essential Fatty Acids

In certain embodiments, the individual's diet cohort is defined, at least in-part, by the individual's consumption of, and requirement for, non-essential fatty acids. In these embodiments, the individual is provided a nutritional supplement and/or program to balance the requirement. Optimal levels for basic dietary cohorts of meat, plant, and seafood heavy diets is disclosed herein (see Tables 6 to 8).

Non-essential fatty acids can be synthesized endogenously, however some of them are considered conditionally essential and they may influence EFA metabolism. For example, OA can have regulatory functions in addition to altering cellular fatty acid composition in select organs. Fatty acids contribute to many cellular functions including homeostasis, coordinating the expression of proteins involved in lipid synthesis, transport, storage, degradation, and elimination to maintain a normal physiological state. Subsequent to meal ingestion lipids in the duodenum regulate energy and glucose homeostasis through a feedback mechanism to the central nervous system which ultimately regulates food intake. This sensitive neuronal circuitry can become defective in response to high-fat or fat imbalance. Certain fatty acids, palmitic, lauric, and stearic, have a role in stimulating the expression of mitochondrial uncoupling proteins, UCP2 and UCP3, which reduce oxidative stress and are associated with longevity.

Not only omega-6 and omega-3 fatty acids but most other fatty acids also compete in metabolic pathways such that dietary fat is reflected in tissue composition. Total amount of dietary fatty acids (low-fat versus high-fat diets) can also influence the fatty acid metabolism and tissue composition. For example, increased omega-3 fatty acid levels in plasma fatty acids from low fat diets have been observed, which is likely due to preferential metabolism of ALA. Other studies have shown that dietary fat quantity outweighs fat type in influencing blood pressure, a risk factor for vascular disease. Thus, omega-6 and omega-3 ratios and amounts should be considered in conjunction with the influencing factors.

### Microorganisms, Prebiotics, Probiotics, Synbiotics

In certain embodiments, the individual's diet cohort is defined, at least in-part, by the individual's consumption of, and requirement for, microorganisms, namely prebiotics, probiotics, and synbiotics. In these embodiments, the individual is provided a nutritional supplement and/or program to balance the requirement.

The nutritional program may include one or more prebiotics and/or fiber (soluble and/or insoluble). The nutritional program may include one or more probiotics. In general, it is believed that these micro-organisms inhibit or influence the growth and/or metabolism of pathogenic bacteria in the intestinal tract. Probiotics may also activate the immune function of the host.

The nutritional program or formulation may include one or more synbiotics, fish oils, and/or phytonutrients. As used herein, a synbiotic is a supplement that contains both a prebiotic and a probiotic that work together to improve the microflora of the intestine.

Gut microflora influences the capacity of an individual to obtain energy from diet. That microflora also influences lipogenesis and plasma lipopolysaccharide levels implicated in inflammation, obesity, and type-2 diabetes. A high-fat diet creates unfavorable gut microflora. Conversely, gut microorganisms influenced fat composition of host tissue. Oral administration of Bifidobacterium breve with linoleic acid increased the tissue composition of conjugated-linoleic acid and omega-3 fatty acids EPA and DHA.

### Oxidation and Antioxidants

In certain embodiments, the individual's diet cohort is defined, at least in-part, by the individual's consumption of, and requirement for, antioxidants. In these embodiments, the individual is provided a nutritional supplement and/or program to balance the requirement. Optimal levels for basic dietary cohorts of meat, plant, and seafood heavy diets is disclosed herein (see Tables 6 to 8). In certain embodiments, the cohort is defined by, and the formulation designed to supplement or withdraw from the diet, one or more of vitamin C, vitamin E, and/or selenium, iron, copper, and/or zinc.

In relation to lipid metabolism, fatty acids may undergo any one of the following after ingestion: (1) primarily mitochondrial and peroxisomal β-oxidation for energy production, (2) free-radical mediated oxidation (chain reactions where one free radical can oxidize many lipid molecules), (3) free-radical independent, non-enzymatic oxidation, or (4) enzymatic oxidation to produce bioactive lipid products such as long-chain fatty acids and eicosanoids. Specific products are formed from each type of oxidation and specific antioxidants are required to inhibit each type of reaction. The nutritional program may include antioxidants. Antioxidants are molecules capable of slowing or preventing the oxidation of other molecules. Non-limiting examples of antioxidants include preventative enzymes such as superoxide dismutase (SOD), catalase, and glutathione peroxidases (GSHpx), vitamin A, carotenoids, vitamin C, vitamin E, selenium, flavonoids, Lactowolfberry, wolfberry, polyphenols, lycopene, lutein, lignan, coenzyme Q10 (CoQ10), glutathione or combinations thereof.

Vitamin E and C work synergistically to protect lipids; vitamin C repairs the alpha-tocopheroxyl radical (vitamin E radical) enabling it to resume its antioxidant function. Vitamin E's antioxidant action can reverse age-associated increase in Cyclooxygenase-2 (COX-2) activity and associated increase in PGE2 synthesis by inhibiting the cofactors; this effect also increases T-cell-mediated immune function. Gamma-tocopherol (γT) form of vitamin E has been found to be a more effective inhibitor of PGE2, LTB4, and tumor necrosis factor-α (TNFα) an inflammatory cytokine than alpha-tocopherol (αT). Vitamin E requirements are partially dependent on PUFA consumption, because PUFA may reduce intestinal absorption of vitamin E while increasing the amount needed for PUFA protection.

Selenium, an important component of Se-dependent glutathione peroxidase (Se-GSHpx) and it functions synergistically with vitamin E as an antioxidant to protect cellular fatty acids and enzymes for eicosanoid production. The metal ions zinc, cadmium, silver, iron, and mercury are inhibitors of Se-GSHpx. GSHpx (both Se-dependent and non-Se-independent). Both copper and zinc play a role in SOD mediated protection of COX, and PG and TX synthetases. Copper status is also associated with Se-GSHpx status in liver and lungs.

Many of the antioxidants, phytochemicals, vitamins, and minerals suppress oxidation of PUFA (though some minerals, such as, iron and copper are pro-oxidants) and PG synthesis, thereby increasing the need for LA or omega-6 family of fatty acids, and reducing the need for or tolerance of omega-3 fatty acids. Reduced oxidation affects the omega-6 family more than the omega-3 family because of preferential metabolism of omega-3 family.

Antioxidants have powerful properties and therefore have a narrow window of healthful effects. Low levels of oxidation products (e.g. lipid peroxidation (LPO) products, free radicals) are necessary for cellular functions. Oxidation of molecules proceed by different pathways. Specific products are formed from each type of oxidation and specific antioxidants are required to inhibit each type of reaction. See Buettner G., Arch Biochem Biophys. 1993; 300: 535-543, incorporated herein by reference in its entirety. Droge W. Free radicals in the physiological control of cell function. Physiol Rev. January 2002; 82(1):47-95.

LPO products in plasma of healthy human subjects are below 1 μM and the molar ratios of LPO products to the respective parent lipids are below 1/1000, that is, below 0.1%. Sublethal concentrations of LPO products induce cellular adaptive responses and enhance tolerance against subsequent oxidative stress through upregulation of antioxidant compounds and enzymes. Such opposite dual functions of LPO products imply that LPO, and oxidative stress in general, may exert both deleterious and beneficial effects in vivo. LPO as well as reactive oxygen and nitrogen species has been shown to play important roles as a regulator of gene expression and cellular signaling messenger. In order to exert physiologically important functions as a regulator of gene expression and mediator of cellular signaling, the formation of LPO products must be strictly controlled and programmed. Niki E. Lipid peroxidation: physiological levels and dual biological effects. Free Radic Biol Med. Sep. 1, 2009; 47(5):469-484.

An excessive and/or sustained increase in reactive oxygen species production has been implicated in pathogenesis of many diseases including cancer, diabetes mellitus, atherosclerosis, neurodegenerative diseases, chronic inflammation, rheumatoid arthritis, ischemica/reperfusion injury, obstructive sleep apnea. However, in a study of lipid and lipoprotein profiles, fatty acid composition, and oxidant-antioxidant status in pediatric attention deficit hyperactivity disorder (ADHD) patients, reduced lipid peroxidation was noted. Similarly, disturbances in the lipid profile, in lipoprotein concentrations and composition, and in oxidant-antioxidant status were observed in pediatric Crohn's disease patients.

### Vitamins and Minerals

In certain embodiments, the individual's diet cohort is defined, at least in-part, by the individual's consumption of, and requirement for, vitamins and minerals. In these embodiments, the individual is provided a nutritional supplement and/or program to balance the requirement. Optimal levels for basic dietary cohorts of meat, plant, and seafood heavy diets is disclosed herein (see Tables 6 to 8). In certain embodiments, the cohort is defined by, and the formulation designed to supplement or withdraw from the diet, one or more of vitamin A, vitamin D, vitamin E, vitamin K, vitamin B12, folic acid or folate, selenium, copper, iron, calcium, magnesium, phosphorus, manganese, potassium, sodium, chloride, and zinc.

Some vitamins and minerals may also have powerful properties, i.e. a narrow window of healthful effects because of their prooxidants/antioxidant potential, and their ability to modulate the antioxidant enzyme expression, among other factors. Some of those are: Vitamin A, Vitamin E (tocopherols), Vitamin B9 (Folic acid, particularly food folate in natural form), Vitamin D, Vitamin E, Selenium, Copper, Zinc. Like phytochemicals, some minerals can act as antioxidants and pro-oxidant depending on levels and complement of other nutrients.

### Dietary Fiber

In certain embodiments, the individual's diet cohort is defined, at least in-part, by the individual's consumption of, and requirement for, dietary fiber. In these embodiments, the individual is provided a nutritional supplement and/or program to balance the requirement. Optimal levels for basic dietary cohorts of meat, plant, and seafood heavy diets is disclosed herein (see Tables 6 to 8). In certain embodiments, the cohort is defined by, and the formulation designed to supplement or withdraw from the diet, one or more of cellulose, starch, glucans, cereal bran, and hydrocolloids.

As used herein, “dietary fiber” refers to indigestible and non-metablizable organic material contained in food. Low calorie bulking agents, such as cellulose, starch, glucans, cereal bran, and hydrocolloids (e.g., xanthan, guar, and alginate), generally are indigestible polymers that can be used in food products. These agents, often referred to as “fiber” or “roughage,” pass through the digestive system for the most part intact and have been shown to have a number of actual and potential health benefits.

Dietary fiber may be divided into predominantly soluble or insoluble fibers (depending on solubility in water). Both types of fiber are present in substantially all plant foods, with varying degrees of each depending on the plant. Water soluble dietary fiber, or “soluble fiber”, refers to dietary fiber that is water soluble or water swellable. Water soluble dietary fibers include, for example, oligosaccharides, psyllium, beta glucan, oat bran, oat groat, pectin, carrageenan, guar, locust beau gum, gum acacia, and xanthan gum, and the like and combinations thereof. Dietary fiber typically consists of non-starch polysaccharides, for example, cellulose and other plant components including dextrins, inulin, lignin, waxes, chitins, pectins, beta-glucans and oligosaccharides.

Dietary fibers affects nutrition by changing the nature of the contents of the gastrointestinal tract, and by changing how other nutrients and chemicals are absorbed. The addition of such indigestible fiber materials to food stimulates the intestine to peristalsis, resulting in increased digestion of accompanying food materials. Due to its effect on digestion, increased consumption of dietary fiber has been linked to decreases in the incidence of gastrointestinal diseases, including bowel cancer. Prebiotic soluble fiber products, like those containing inulin or oligosaccharides, may contribute to relief from inflammatory bowel disease, as in Crohn's disease, ulcerative colitis, and Clostridium difficile, due in part to the short-chain fatty acids produced with subsequent anti-inflammatory actions upon the bowel. Consistent intake of fermentable fiber through foods like berries and other fresh fruit, vegetables, whole grains, seeds and nuts is now known to reduce risk of several diseases—obesity, diabetes, high blood cholesterol, cardiovascular-disease, bowel cancer, and numerous gastrointestinal disorders including irritable bowel syndrome, diarrhea, and constipation.

### Gender

In certain embodiments, the individual's diet cohort is defined, at least in-part, by the individual's gender. In these embodiments, the individual is provided a nutritional supplement and/or program customized for gender.

While sex hormones can alter metabolism of dietary fats, dietary fats can alter synthesis of sex hormones and the associated receptor organization. Increasing amount of dietary fat increases the androgen production, depending on the fatty acids administered. Higher PUFA administration resulted in lower activity of steroidogenic enzymes and lower levels of androgens as compared to MUFA or SFA administration. Omega-3 fatty acids, particularly DHA caused less androgen production than omega-6 fatty acids; and omega-6 fatty acids caused less androgen production than MUFA or SFA. The period over which the dietary fat was fed to an animal also altered androgen levels; initially sharp increases correlated with the dietary levels after 3 weeks, followed by significant reductions after 6 weeks, demonstrating an adaptation mechanism. The response may be a homeostatic adjustment possibly due to LCPUFA's similar actions and benefits as sex hormones. Though the relationship is not well understood yet, parallels have been drawn to estrogen. Both estrogen and PUFA enhance nitric oxide synthesis, suppress the production of pro-inflammatory cytokines, show antioxidant-like and anti-atherosclerotic properties, and have neuroprotective actions. The relationship of fatty acids with androgens also has significance for men. High levels of androgens may be associated with carcinogenesis, while low levels may be deleterious to semen quality. Men and women also differ in storage, mobilization, and oxidation of fatty acids, and gene expression relevant to fatty acid metabolism.

### Genetics

In certain embodiments, the individual's diet cohort is defined, at least in-part, by the individual's genetic polymorphisms, and/or consumption and requirement for methyl donor compounds. In these embodiments, the individual is provided a formulation tailored to the individual's genetics, or requirement for methyl donor nutrients. In certain embodiments, the cohort is defined by, and the formulation designed to supplement with one or more of folate, vitamin B-12, vitamin B-6, choline, methionine, genistein, coumesterol, and polyphenol. Taking into account the known existence of genetic polymorphisms, the individual's diet may be supplemented with or restricted of certain phytochemicals including one or more of curcumin, capsaicin, ginsenosides, hesperidin, and resveratrol.

Genetic code, the sequence of nucleotides in our DNA, can influence health status. But there is another set of instructions that affect gene expression, and this set of instructions can be altered by diet. Epigenetics, the study of heritable changes in gene function that occur independent of a change in DNA sequence, represents a new frontier in biomedical science that has important implications for dietetics practice. For example, one way in which gene expression is modulated is through DNA methylation—the degree to which methyl groups are present or absent from certain regions of our genes. Depending on the circumstances, hypomethylation or hypermethylation can be beneficial or harmful depending on which genes are turned on or off, at what point in time, and in which tissues. DNA methylation can be affected by intake of folate, vitamins B-12 and B-6, choline, and methionine because these nutrients are involved in the generation of methyl groups through one carbon metabolism. Other dietary factors, such as genistein, coumesterol, and polyphenols also influence DNA methylation. Stover P J, Caudill M A. Genetic and epigenetic contributions to human nutrition and health: managing genome-diet interactions. J Am Diet Assoc. 2008; 108:1480-1487. Barnes S. Nutritional genomics, polyphenols, diets, and their impact on dietetics. J Am Diet Assoc. 2008; 108:1888-1895.

Genetic variations can also influence the metabolism and therefore requirement of lipids. Polymorphisms in apolipoprotein E and peroxisome-proliferator-activated receptor-gamma (PPARγ) genes may influence response to dietary fats. However, dietary fats can alter many genes. PUFA suppress lipogenic, glycolytic, and choelsterolgenic genes, but increase expression of genes for enzymes needed in the β-oxidation pathway. Simopoulos A P. The role of fatty acids in gene expression: health implications. Ann Nutr Metab. 1996; 40:303-311. Sampath H, Ntambi J M. Polyunsaturated fatty acid regulation of genes of lipid metabolism. Annu Rev Nutr. 2005; 25:317-340. PUFA modulate gene expression by interacting with nuclear receptor hepatic nuclear factor (HNF-4), liver X receptors (LXR), and PPAR α, β, δ, and γ, and by regulating the transcription factor sterol regulatory element-binding proteins (SREBP) 1 & 2. SREBP, suppressed by PUFA, are key regulators of cholesterol, fatty acid, and triglyceride synthesis. LA and AA are potent PPAR ligands, producing rapid increase in expression of genes involved in lipid oxidation.

Phytochemicals may also influence the expression of a range of genes. Several phytochemicals can bind to cell surface and nuclear receptors as ligands. Curcumin, capsaicin, ginsenosides, hesperidin, and resveratrol are known PPARγ ligands, believed to attenuate cytokine production and inflammation. Phytosterols can also alter intestinal and liver gene expression. Since nutrients can change gene expression, it is more effective to design nutrition (fewer variables, greater control, and easier implementation) for optimum gene-expression, rather than nutrition for disease states caused by unhealthy nutrition.

### Aging

In certain embodiments, the individual's diet cohort is defined, at least in-part, by the individual's age. In these embodiments, the individual is provided a formulation tailored to the individual's age. In certain embodiments, the cohort is defined by, and the formulation designed to supplement with one or more antioxidants, fatty acids, and phytosterols.

Aging brings about a decline in sex hormones, increased oxidative stress, and decreased homeostatic regulation and immunity. Oxidative stress is currently one of the most accepted theories of aging, where aging is the result of lifelong and progressive damage to molecules from oxidation products and the consequential deterioration of physiological functions. Hulbert A J, Pamplona R, Buffenstein R, Buttemer W A. Life and death: metabolic rate, membrane composition, and life span of animals. Physiol Rev. October 2007; 87(4):1175-1213. Since fatty acids are the molecules most vulnerable to oxidation, membranes with fatty acid compositions least prone to lipoxidative damage are associated with longevity. Fatty acids differ dramatically in their susceptibility to peroxidation. Birds, who have exceptionally long lifespan relative to their body mass favor lower unsaturation index of omega-6 PUFA to higher unsaturation index of omega-3 PUFA in membranes.

PUFA and unsaturation index have been shown to increase with advancing age in most tissue except for brain where they decline; but membrane fluidity declines uniformly with age because of peroxidation and possibly altered fatty acid chain composition. Unsaturated fatty acids are said to contribute to fluidity. Oxidized lipids and LPO are greater cause for membrane rigidity than low unsaturation index. Antioxidants are unable to increase the maximum life span of a species, but they have been shown to increase mean life span in select populations.

A decline in brain PUFA, particularly DHA, with age has been shown to be associated with increased lipid peroxidation. A decline in cognitive function along with neuronal apoptosis of cerebral cortex and hippocampus has also been found to be associated with age or hyperoxia, and prevented by vitamin E. Since an aging brain has been shown to have lower DHA, fish oils have been suggested to increase tissue DHA levels because they bypass D6D and D5D and directly provide long-chain omega-3 fatty acids in form of EPA and DHA. However, dietary fish oils rich in DHA and EPA strongly suppress D6D, with implications for other LCPUFA levels. Cho H P, Nakamura M, Clarke S D. Cloning, expression, and fatty acid regulation of the human delta-5 desaturase. J Biol Chem. Dec. 24 1999; 274(52):37335-37339.

As noted before, LCPUFA increase in tissue other than the brain with age, which may be a compensation for decline in hormones because of similarities in actions. Studies with rats have demonstrated lower desaturase activity with age, which may be reversed with GLA. However, GLA was significantly more effective on DHA than on AA restoration. Therefore, reduced AA levels with age may be of concern, particularly in women and vegetarian women. The solution might lie in optimal mix of fatty acids and antioxidants with phytosterols, which increase desaturase activity and have antioxidant properties and hormone-like actions, such that greater membrane fluidity and lower unsaturation index can be achieved.

### Temperature

In certain embodiments, the individual's diet cohort is defined, at least in-part, by the individual's climactic temperature. In these embodiments, the individual is provided a formulation tailored to the individual's climactic temperature. In certain embodiments, the cohort is defined by, and the formulation designed to supplement with optimal dietary lipids and phytochemicals.

In general, a greater unsaturation index of fatty acids occurs in tissues at lower temperatures in order to maintain homeoviscosity and optimal membrane and cellular functions. Increased unsaturation preserves function at low temperature and decreased unsaturation preserves function at high temperature, but excessively low PUFA levels also reduce heat tolerance. Although membrane lipid composition is the main acclimatory response to changes in climactic temperature, other responses may include altered expression of membrane proteins, altered composition of bilayer stabilizing versus destabilizing lipids, and altered proportions of plasmalogens compared to diacyl phospholipids. Phytochemicals can also alter membrane properties including fluidity. Thus, while the body adapts to changes in temperature, benefit can be derived by customizing dietary lipids and phytochemicals with respect to temperature, such that raw materials conducive to self-regulation are present in optimal quantities.

Inflammatory Pathways—Relationship with Nutrients

In certain embodiments, the individual's diet cohort is defined, at least in-part, by the individual's inflammatory state. In these embodiments, the individual is provided a formulation tailored to the individual's inflammatory state. In certain embodiments, the cohort is defined by, and the formulation designed to supplement or withdraw, dietary phytochemicals, antioxidants, vitamins, and minerals, such as one or more of flavonoids, sulforaphane, curcumin, and zerumbone, capsaicin, ginsenosides, hesperidin, and resveratrol, omega-3, omega-6 (including the omega-6:omega-3 ratio), In some embodiments, the phytochemicals include one or more of procyanidins, epigallocatechin gallate, epicatechin 3-gallate, resveratrol, apigenin, luteolin, quecetin, anthocyanins and hydrocinnamic acids, curcumin, hesperidin, diosmin, amentoflavone, bilobetin, morelloflavone, ginkgetin, and yuccaols A, B, C, D and E. In some embodiments, the phytochemicals are as defined in Table 1 and 3. The diet cohort may be defined by the level of consumption of the sources disclosed in Table 1 and 3, and the nutritional program may be tailored by supplementation or withdrawal of these sources. Some phytochemicals, antioxidants, vitamins and minerals interactions can lead to harmful health effects. Phytochemicals and antioxidants can suppress a number of inflammatory pathways. Excessive suppression may be problematic in that some inflammation may be necessary, and that compensatory mechanisms may be put in motion. Phytochemicals, particularly flavonoids have been found to have antimicrobial, antiviral, anti-ulcerogenic, cytotoxic, antineoplastic, mutagenic, antioxidant, antihepatotoxic, antihypertensive, hypolipidemic, anti-aging, antiplatelet and anti-inflammatory activities. They also have biochemical effects, which inhibit a number of enzymes such as aldose reductase, xanthine oxidase, phosphodiesterase, Ca+2-ATPase, lipoxygenase, cycloxygenase, etc. Additionally, they also have a regulatory role on different hormones like estrogens, androgens and thyroid hormone.

Excessive phytochemicals, inadequate or imbalanced lipids and/or their impaired metabolism, and/or their interactions may dysregulate cytokines involved in inflammation: TGF-β1, TNF-α, IL-1β, IL4, IL5, IL6, IL8, IL10, IL13, and γ-IFN. Particular diseases that may be implicated are disorders of the immune system, for example systemic lupus erythematosus (SLE), allergy, asthma, Crohn's disease and rheumatoid arthritis, but particularly multiple sclerosis, and also neurodegenerative diseases such as sequelae of stroke, head trauma, bleeds, Alzheimer's and Parkinson's disease, and sepsis, coronary heart disease (CHD), and infant abnormalities.

LCPUFA play an important role in inflammation and immunity. At low levels AA augments or is neutral to certain immune function, but at high levels it has an inhibitory effect. Intakes of long-chain omega-3 appear to be inhibitory on a wide range of immune functions: autoantibody production, T-lymphocyte proliferation, apoptosis of autoreactive lymphocytes, and cytokines and leukotrienes. Many of the effects of long-chain omega-3 appear to be due to the inhibition of transcription factor, NFkB, which regulates broad range of cytokine genes involved in inflammation. Calder P C. Polyunsaturated fatty acids and inflammatory processes: New twists in an old tale. Biochimie. June 2009; 91(6):791-795. Long-chain omega-3 also activate transcription factor PPARγ, which can modulate anti-inflammatory genes and inhibit NFkB. Therefore, in the short run omega-3 may ameliorate the symptoms of diseases associated with low-grade inflammation; but in the long run they may compromise host immunity. Further, the effects may be compounded by certain phytochemicals which also inhibit NFkB (e.g. sulforaphane, curcumin, and zerumbone) or activate PPARγ (e.g. curcumin, capsaicin, ginsenosides, hesperidin, and resveratrol). Sudden and wide fluctuations in phytochemicals and or fatty acids intake can change the immune response and rest of the physiology. Withdrawing a phytochemical or Omega-3 or any immunosuppressive/inflammation suppressive nutrient may unleash excessive inflammation.

Depending upon fatty acids and phytochemical tissue stores, a sudden withdrawal of a habitual high long-chain omega-3 fatty acids or immunosuppressive or antiinflammatory phytochemical supply from the host, or a sudden increase in omega-6 fatty acids or other fatty acids may result in unrestrained cytokine response, with severe consequences involving systemic inflammatory response (capillary leakage, pyrexia, tachycardia, tachypnoea), multi-organ dysfunction (gastrointestinal, lungs, liver, kidney, heart), and joint tissue damage. In addition to sudden increases in cytokine action, other factors such as sudden change in excitability of neural and muscle cells may be another complicating factor. At such instances the host may become vulnerable to infections, myocardial infarction, stroke, and induction of psoriasis depending upon the rest of the body chemistry and the presence of infectious agents. In less severe manifestations, due to moderate fluctuations in fatty acids and in otherwise salubrious condition, the host may experience sleep disturbances, headaches, muscle cramps, confusion, melancholia, and rage resulting from changes in neurotransmission, excitability of muscle and neural cells, and fluctuating eicosanoids and androgens. As a nutritional strategy, cumulative effects of all dietary inflammation modulation should be below the threshold where self-regulation of the immune system is materially blunted or inflammation is dysregulated.

Different flavonoids display anti-inflammatory mechanisms of action. For example (from Rathee et al. Mechanism of Action of Flavonoids as Anti-inflammatory Agents: A review. Inflammation & Allergy-Drug Targets, 2009, 8, 229-235.)

Procyanidins—Inhibits transcription and secretion of IL-1

Epigallocatechin gallate—Inhibits the expression of iNOS—Reducing the activity of NF-kB and AP-1.

Epicatechin 3-gallate—Attenuates adhesion and migration of peripheral blood CD8+T cells.

Resveratrol—Inhibits stimulation of caspase-3 and cleavage of PARP induced by IL-1alpha. Suppressing the expression of iNOS mRNA and protein by inhibiting the activation of NF-kB Inhibiting NO generation. Upregulating MAP kinase phosphatase-5

Apigenin—Blocks the expression of intercellular adhesion molecule-1 (ICAM-1), VCAM-1, and E-selectin. Inhibiting prostaglandin synthesis and IL-6, 8 production.

Luteolin—Inhibits the upregulation of THP-1 adhesion and VCAM-1 expression. Inhibiting the activity of the NF-kB.

Quercetin—Inhibits NO production and iNOS protein expression. Inhibits both cyclooxygenase and lipoxygenase activities.

Anthocyanins & Hydroxycinnamic acids—Localizes into endothelial cells. Reducing the upregulation of IL-8, MCP-1, and ICAM-1

Curcumin—Decreases MPO activity and TNF-alpha on chronic colitis. Reducing nitrites levels and the activation of p38 MAPK. Downregulating COX-2 and iNOS expression.

Hesperidin, Diosmin—Inhibits prostaglandin formation.

Amentoflavone, Bilobetin, Morelloflavone, Ginkgetin—Inhibits phospholipase C1 and A2.

Yuccaols A, B, C, D and E—Inhibit the nuclear transcription factor NF-kB.

The activities of the nutrients may be mediated by interactions with one or more of cell-derived mediators (listed in Table 2, from Rathee et al.).

TABLE 2
Cell-derived mediators

| Name | Type | Source | Description |
|---|---|---|---|
| Lysosome granules | Enzymes | Granulocytes | contain a large variety of enzymes which |
| | | | act as inflammatory mediators |
| Histamine | Vasoactive | Mast cells, | Stored in preformed granules, histamine |
| | amine | basophils, | is released in response to a number of |
| | | platelets | stimuli |
| IFNgamma | Cytokine | T-cells, NK | Antiviral, immuno-regulatory, and anti- |
| | | cells | tumour properties. This interferon was |
| | | | originally called macrophage-activating |
| | | | factor, and is especially important in the |
| | | | maintenance of chronic inflammation |
| IL-8 | Chemokine | Macrophages | Activation and chemo-attraction of |
| | | | neutrophils, with a weak effect on |
| | | | monocytes and eosinophils |
| Leukotriene B4 | Eicosanoid | Leukocytes | Mediates leukocyte adhesion and |
| | | | activation. In neutrophils, it is also a |
| | | | potent chemo-attractant, and induces the |
| | | | formation of reactive oxygen species and |
| | | | the release of lysosome enzymes by these |
| | | | cells. |
| Nitric oxide | Soluble gas | Macrophages, | Potent vasodilator, relaxes smooth |
| | | endothelial | muscle, reduces platelet aggregation, aids |
| | | cells, some | in leukocyte recruitment, direct |
| | | neurons | antimicrobial activity in high |
| | | | concentrations. |
| Prostaglandins | Eicosanoid | Mast cells | A group of lipids which cause |
| | | | vasodilation, fever, and pain. |
| TNFalpha & IL-1 | Cytokines | Macrophages | Affect a wide variety of cells to induce |
| | | | inflammatory reactions: fever, |
| | | | production of cytokines, endothelial gene |
| | | | regulation, chemotaxis, leukocyte |
| | | | adherence, activation of fibroblasts. |

Several nutrients are known to have cardiovascular disease (CVD) related activities as shown in Table 3 (from M. Massaro et al., Nutraceuticals and Prevention of Atherosclerosis; Cardio vascular Therapeutics; 28 (2010), Table 2)

TABLE 3
Known CV disease related actions of different nutrients

| Bioactive | | | |
|---|---|---|---|
| compound | Examples | Sources | Putative effects |
| Flavonols | Quercetin, | Onion, apple, tea, | ↓TC, ↓LDL-C oxidation↑HDL-C, |
| | kaempferol, | berries, olives, | AOx, ↓platelet aggregation, |
| | catechin | broccoli, lettuce, | ↓eicosanoid synthesis, ↓athero- |
| | | red wine, | ELAMs, ↓angiogenesis, ↓MMPs |
| | | cocoa/chocolate | |
| Flavonols | Epicatechin, | Green/black tea, | AOx, ↓apoptosis, ↓LDL-C oxidation, |
| | epigallocatechin, | cocoa/chocolate | ↓platelet aggregation, ↓athero- |
| | epicatechin-3- | | ELAMs, ↓angiogenesis, ↓MMPs |
| | gallate, | | |
| | epigallocatechin- | | |
| | 3-gallate | | |
| Lignans | Enterolactone, | Flaxseed oil, | ↓LDL-C, AOx, |
| | enterodiol | lucerne, clover | estrogen/antiestrogen; |
| | | | ↓atherosclerosis in vivo but may |
| | | | show adverse CVD effect (pro- |
| | | | oxidant activity with partially |
| | | | defatted flaxseed) |
| Isoflavones | Genistein, | Soybeans, legumes | ↓TC and LDL-C, ↓LDL-C oxidation, |
| | daidzein | | ↓TG, ↑HDL-C, |
| | | | ↓thrombosis, AOx, |
| | | | estrogen/antiestrogen, ↓athero- |
| | | | ELAMs, ↓angiogenesis, |
| | | | ↓atherosclerosis in vivo, ↓MMPs |
| Stilbenoids | Resveratrol | Grapes, red wine, | ↓LDL-C oxidation, ↓platelet |
| | | peanuts | aggregation/thrombosis, ↓eicosanoid |
| | | | synthesis, AOx, ↓athero-ELAMs, |
| | | | ↓angiogenesis but promotes |
| | | | angiogenesis in the ischemic |
| | | | heart, ↓atherosclerosis in vivo, |
| | | | ↓MMPs |
| Carotenoids | Lycopene | Tomatoes, tomato | ↓LDL-C and LDL-C oxidation, AOx, |
| | | products | ↓athero-ELAMs, ↓MMPs, but no |
| | | | effects was shown in animal models |
| | | | of ATS and dietary intervention |
| | | | studies using well-defined subjects |
| | | | population did not provided a clear |
| | | | evidence of lycopene in the |
| | | | prevention of CVD |
| Carotenoids | α-Carotene, β- | Carrots, pumpkins, | Inconsistent data. β-carotene has |
| | carotene, γ- | maize, tangerine, | shown adverse CVD effect because |
| | carotene, δ- | orange and yellow | its prooxidant activity |
| | carotene | fruits and | |
| | | vegetables | |
| Organosulfur | Allicin, diallyl | Garlic, onion, leek | ↓TC and LDL-C, ↓TG, ↓cholesterol |
| compounds | sulfide, diallyl | | and FA synthesis, ↓BP, ↓thrombosis, |
| | disulfide, allyl | | AOx, ↓athero-ELAMs, |
| | mercaptan | | ↓angiogenesis, |
| | | | ↓atherosclerosis in vivo, ↓MMPs |
| Soluble dietary | Glucan, pectin | Psyllium, oats, | ↓TC, ↓TG, ↓LDL-C |
| fibers | | barley, yeast, fruit, | |
| | | vegetables, | |
| | | psyllium seed, | |
| | | fortified cereals | |
| | | and grains | |
| Isothiocyanates | Phenethyl | Cruciferous | no relevant effects |
| | (PEITC), benzyl | vegetables (e.g., | |
| | (BITC), | watercress, | |
| | sulforaphanes | broccoli) | |
| Monoterpenes | d-Limonene, | Essential oils of | ↓TC and LDL-C, ↓HMGCoAR, |
| | perillic acid | citrus fruit, | ↓angiogenesis |
| | | cherries, mint, | |
| | | herbs | |
| Plant sterols | Sitostanol, | Tall oil, soybean | ↓TC and LDL-C, AOx, ↓cholesterol |
| | stigmasterol, | oil, rice bran oil | absorption; |
| | campesterol | | adverse effect: ↓carotenoid |
| | | | absorption |
| Phenolic acids | Tyrosol, | Extra virgin olive | ↓LDL-C oxidation, ↓platelet |
| | hydroxytyrosol, | oil | aggregation/thrombosis, ↓eicosanoid |
| | oleoeuropeine, | | synthesis, AOx, ↓athero-ELAMs, |
| | caffeic acid, | | ↓atherosclerosis in vivo, ↓MMPs |
| | cumaric acid | | |
| ω-3 PUFA | DHA, EPA, αLA | Fish and fish oil, | ↓TC, suppression of cardiac |
| | | green leaves | arrhythmias, ↓BP |
| | | | ↓platelet aggregation, ↓eicosanoid |
| | | | synthesis, ↓athero-ELAMs, |
| | | | ↓angiogenesis; ↓MMPs |
| Prebiotics | Inulin-type | Fruit and | ↓TC and ↓TG |
| | fructans | vegetable, purified | |
| | | extract from | |
| | | chicory root | |
| Probiotics | Selected strains | Fermented milk | ↓TC, LDL-C and BP |
| | of Lactobacillus | products | |
| | acidophilus, | | |
| | Bifidobacterium | | |
| | bifidum and | | |
| | Lactobacillus | | |
| | bulgaricus | | |

AOx, antioxidant activity;
BP, blood pressure;
CVD, cardiovascular disease;
HDL-C, high density lipoprotein cholesterol;
LDL-C, low density lipoprotein cholesterol;
TC, total cholesterol;
TG, triglycerides;
MMPs, metalloproteinases;
ELAMs, endothelial leukocyte adhesion molecules.

### Health Effects of Whole/Natural Food Items

Whole foods have different health effects than one would predict from the sum of the parts. Nutrients have different properties in one form versus another, e.g. conjugated versus free. This is due to alteration in metabolism, presence and/or composition of other nutrients and/or absorption; nature of connection between nutrients and context is crucial. Flavonoids which are mainly present as glycosides in food (with the exception of catechins) are expected to be poorly absorbed, but quercetin glycosides are absorbed in appreciable amounts in the small intestine. For instance, the flavonoid quercetin was shown to be more bioavailable as an aglycone than quercetin glucosides when ingested as onion flesh, while quercetin glycosides where more available when ingested as dried onion skin. Beneficial or harmful effects of nutrients including phytochemicals can be explained by additive and synergistic effects, as vegetables and fruits contain multiple different phytochemicals which seem to influence and potentiate each other. Synergistic effects increase bioavailability. For example quercetin is an inhibitor of resveratrol sulfation in the liver and small intestine and increases the bioavailability of resveratrol. The synergistic effect of piperine on curcumin is driven by its inhibiting effect on curcumin conjugation. Further, absorption of phytochemicals can be enhanced by complexing with lipids or by nanoparticles that increase the water solubility of hydrophobic drugs.

### Development of Novel Dietary Programs

The invention relates to development of nutritional compositions and/or formulations that balance phytochemicals, antioxidants, vitamins, minerals, acid-base, lipids, proteins, carbohydrates, probiotics, prebiotics, microorganisms, fiber, and the like. Levels and types of nutrients in each food item are considered in developing a nutritional plan that provides nutrients at levels that have exemplary health benefits. In some aspects, nutritional plans are tailored to fit the primary dietary preferences of consumers.

To be effective, the nutritional plans are designed such that at least 25%, 50%, 60%, 70%, 80% or 100% of calories in the diet are provided by the foods specified in the plan over an extended period of time.

In one aspect, packages and kits are provided to support specific aspects of the nutritional plan. In some embodiments, the packages and kits comprise component or modular systems comprising vegetable, fruit, grains, cereals, legumes, meats, seafood, nuts, seeds, herbs, lipids, milks, yogurts and the like, and any combination thereof. In some embodiments, the packages and kits comprise unprepared, or ready to cook foods, such as fruit, vegetable, legume, dry grain, meat, seafood, herbs, fat, nuts and seeds, milks, yogurts, and the like. In some embodiments, the packages and kits comprise processed or cooked foods such as a nutritional bar; a bakery food product such as a bread, a dessert, a pastry, a truffle, a pudding or cake; a salad, a drink, a yogurt, a milk, a side dish, a snack, a meal; a gel, a puree, a sauce, a dressing, a spread, a butter, drops, or the like; a sealed single dosage packet or resealable packaging containing a liquid, semi-liquid, semi-solid, or a solid. In some embodiments, they may be unsealed and taken orally, or added as part of a cooking ingredient to previously cooked or uncooked food preparation with or without added fat. For example, they can be made into an oil blend, or a special cooking oil such as a frying oil, a pan-frying oil, a parting oil or the like. The components of the compositions or formulation may be delivered in one-part or multiple parts as various components of a meal or to complement a meal, for example.

In some embodiments, the kits and packages comprise food suitable for consumption by babies and include, but are not limited to soybean-based formula, milk formula, standard milk formula, follow-on milk formula, toddler milk formula, hypoallergenic milk formula, prepared baby food, dried baby food and other baby food.

In some embodiments, the compositions/formulation disclosed herein may be administered to an individual in any orally accepted form. In some embodiments, they may be part of an enteral or parenteral formula, or a combination thereof. In some embodiments, they may be administered topically via a liquid, cream or patch formulation. The formulations may be packaged in one, two, three, four or more mutually complementing daily dosages. In some embodiments, they may be contained in any one or more of, but not limited to, a single dosage or sustained and controlled release capsule, soft-gel capsule, hard capsule, tablet, powder, lozenge, or pill prepared in some instances with carriers such as starches, sugars, diluents, granulating agents, lubricants, binders, disintegrating agents, a granule, and the like. In some embodiments, the compositions may be delivered using a gelatinous case, a vial, a bottle, a pouch or a foil, or plastic and/or card-board box, and the like, or a combination thereof for containing such compositions. In some embodiments a one-day, one-week, two-week, bi-weekly, bi-monthly, or monthly diet plan may be formulated comprising various formulations described herein, with varying compositions administered each day.

In some embodiments, each pack contains specific nutritional content suitable for a balanced and optimized diet. For example, a grain or cereal pack may contain polyphenols, antioxidants, omega fatty acids and/or saturated fatty acids within specific ranges wherein each range is suitable for a specific dietary cohort. Likewise, a fruit, legume or vegetable pack or drink package may be similarly classified. In some embodiments, each pack comprises identification of the ranges of specific critical nutrients and nutrients. In some embodiments, each pack or module is identified by the specific dietary cohort it is suitable for. In some aspects, each module can effectively fit into a nutritional plan when each component or module individually provides less (or greater) than 100, 200, 300, 400, or 500 calories and/or less (or greater) than 10%, 20%, 25%, 30%, or 40% percent of an individual's daily caloric need.

In some aspects, the formulations described herein have high antioxidant and phytochemical content and properties that render extra omega-3 unnecessary, or enhance bioavailability, and/or endogenous synthesis of long-chain omega-3. In specific embodiments, nuts, legumes, grains, sweeteners (such as honey), and herbs/spices (such as curcumin) included in the compositions can render extra omega-3 unnecessary.

In one aspect, food items recommended in a diet plan or contained in a specific component or module are selected based on the methods of processing or manufacturing used in preparing the food items such that optimal nutrient is achieved, and/or desired activation or inactivation of nutrients is achieved. Such processes include steps in preparing the food items such as hulling, removing a layer or part, peeling, drying versus providing fresh or frozen, and method of cooking such as soaking, sprouting, grinding, roasting, baking, grilling, heating, sautéing, fermenting, and the like. Method of processing (removing a layer, cooking, grinding, roasting, soaking, dry versus fresh) is selected to arrive at a formulation from different sources wherein nutrients complement each other. Different parts of plants may contain different strengths of phytochemicals and antioxidants. For example, seed (ovule of flowering plant or part thereof), leaves, stems, flowers or fruits; and skin versus flesh. Seeds include edible kernel, endosperm, germ, and bran or husk. Removing a layer, cooking, grinding, roasting, soaking, dry versus fresh or frozen can change the strength of nutrients.

In one aspect, the invention relates to developing a tailored dietary program and optimizing levels of dietary nutrients therein. Different programs are developed according to general dietary preferences. In general, individual consumers have specific preference for the main foods they like to consume, for example, high or low plant foods versus high or low red meats versus high or low seafood. Henson S, Blandon J, Cranfield J, Herath D. Understanding the propensity of consumers to comply with dietary guidelines directed at heart health. Appetite. 2010; 54:52-61. Diets rich in legumes, fruits, vegetables, whole grains, herbs, nuts and seeds are inherently high in antioxidants and phytochemicals. Grains, vegetables, fruits, legumes, herbs, nuts and seeds are the richest source of phytochemicals and antioxidants. (Halvorsen et al. J. Nutr. 2002; 132:461-471). Mazur W. Phytoestrogen content in foods. Baillieres Clin Endocrinol Metab. 1998; 12:729-742. Consumers are categorized into dietary cohorts according to average amount of the main foods consumed. Commonly consumed foods are not that many. There are a limited number of grains, vegetables, fruits, herbs, meats, seafoods, drinks, and sweeteners. Commonly consumed foods are so because of their nutritive value, safety proven over centuries, and ease of cultivation.

In some aspects, dietary cohorts are based on basic dietary habits and amount of plant foods, meats, and/or seafood in diet. These preferences determine bulk of the food consumption. For example vegetarians are predisposed to eating a lot of and certain kinds of phytochemicals and antioxidants. They may depend on legumes to meet their protein requirement, which inherently increases consumption of flavonoids, and certain kinds of proteins, which affects their requirement for other nutrients. Similarly, seafood inherently includes significant amounts of omega-3, and selenium. Similarly, high meat consumers are inherently and consistently consuming certain kinds of proteins and are deficient in certain phytochemicals and antioxidants. Basic dietary habits can help establish average nutrients consumed from the most commonly consumed major foods. Diet plans may be developed for and around such cohorts. Once the bulk of foods consumed or should be consumed by such cohorts is established, then complementing lipids, phytochemicals, antioxidants, vitamins, minerals, and microorganism programs/formulations are determined based on what achieves the best outcomes. Such a program may reduce the probability of overconsumption or under consumption of critical nutrients. Once nutrient requirement is met and balanced satiety may be achieved. Morton G J, Cummings D E, Baskin D G, Barsh G S, Schwartz M W. Central nervous system control of food intake and body weight. Nature. 2006; 443:289-295.

A method for developing a tailored dietary nutrition program can comprise the following steps: (a) classifying dietary cohorts based on the primary source of calories in a preferred diet of the cohort, or the most common food group in a weekly diet or the types of nutrients in a diet, or inclusion sensitive foods such as seafood; (b) computing the typical range of major nutrients and nutrients in each dietary cohort; (c) preparing a list of food items that fit the dietary preference of the cohort and provide optimized and balanced levels of nutrients; and (d) generating a nutritional plan for consumption over an extended period of time of at least 1, 3, 5 days or 1, 2, 4, 6, 8, or 12 weeks. The plan may be developed for the entire diet or a component thereof, such as lipids. A schematic is provided in Table 4.

In one embodiment, the tailored dietary program is developed by first classifying a subject into a dietary cohort. The range of nutrients in the dietary cohort of the subject is then determined. Finally, a tailored recommended dietary program is developed by determining the dietary nutrients that need to be supplemented or replaced in the diet in order to complement the regular dietary intake of the cohort and achieve optimized nutritional levels.

The method comprises the steps disclosed in Table 4.

TABLE 4
Schematic representation for developing tailored dietary
programs and for optimizing dietary nutrients

| | | |
|---|---|---|
| High | High | High |
| phytochemicals | meat | seafood |

Step 1. Develop dietary cohorts^{a,b}

| | | | |
|---|---|---|---|
| Grains | | | |
| Brown | --to-- cups/g | --to-- cups/g | --to-- cups/g |
| Rice | | | |
| Whole | --to-- cups/g | --to-- cups/g | --to-- cups/g |
| Wheat | | | |
| Other | --to-- cups/g | --to-- cups/g | --to-- cups/g |

| | |
|---|---|
| Vege- | Develop ranges as above |
| tables | |
| Fruits | Develop ranges as above |
| Legumes | Develop ranges as above |
| Dairy | Develop ranges as above |
| Meats | Develop ranges as above |
| Seafood | Develop ranges as above |
| Herbs | Develop ranges as above |
| Sweeten- | Develop ranges as above |
| ers | |
| Beverages | Develop ranges as above |

Step 2. Compute range of nutrients

| | | | |
|---|---|---|---|
| Lipids | | | |
| C4:0 | --to-- mg | --to-- mg | --to-- mg |
| C22:6 Ω3 | --to-- mg | --to-- mg | --to-- mg |
| Other | --to-- mg | --to-- mg | --to-- mg |

| | |
|---|---|
| Carbohy- | Compute ranges as above |
| drates | |
| Protein | Compute ranges as above |
| Vitamins | Compute ranges as above |
| Minerals | Compute ranges as above |
| Phyto- | Compute ranges as above |
| chemicals | |
| Antioxi- | Compute ranges as above |
| dants | |

Step 3. Develop nutritional programs/formulations

Develop programs/formulations to complement the nutrients above, from
natural oils, nuts, seeds, and herbs; additional vitamins and minerals may
be used. Deliver as diurnal mutually complementing individual dosages;
daily variety may strengthen compliance.

| | | | | | | | |
|---|---|---|---|---|---|---|---|
| Monday | Oil blend-A | + | sauce-A | + | spread-A | + | dessert-A |
| Tuesday | Oil blend-B | + | sauce-B | + | spread-B | + | dessert-B |
| Other days | Oil blend-X | + | sauce-X | + | spread-X | + | dessert-X |

^{a}Based on average daily consumption.
^{b}Further customizations may address age, gender, climactic temperature, and medical conditions/lipid tolerance.

Similar cohorts can be defined by age, gender, genetic profile, climactic temperature, and medical conditions such as lipid tolerance. In case of infants, formulations and diet plans may be defined based on mother's diet, genetic profile, and/or medical conditions. In some embodiments, a feedback system is used to fine tune the dietary program according to results achieved.

Dietary cohorts can be based on main foods preferred in the diet of an individual or a group. For example: (a) vegetable based comprising 2, 3, 4, 6, or more servings per day of herbs, legumes, fruits, and vegetables; (b) seafood based comprising 1, 2, 3, 4, 6, or more servings per week of seafood; (c) meat based comprising 3, 4, 6, 8, 10, 12, 14 or more servings per week of meat (red meat) and less than 2, 3, 4 or 6 servings per day of herbs, legumes, fruits, and/or vegetables.

Dietary cohorts can also be defined based on folate, polyphenols, phytosterols, antioxidants, vitamin A, E, Se in the diet. For example, one or more polyphenols greater (or less) than 5, 10, 15, 20, 45, 70, 95, 115, 140, or 165 mg/day; and/or folate greater (or less) than 100, 200, 300, 400, 500, 600, 700, 800, 900, or 1000 mcg/day; and/or one or more phytosterols greater (or less) than 150, 200, 250, 300, 350, 450, 550, 650, 750, or 850 mg/day; and/or Se greater (or less) than 5, 10, 15, 20, 35, 55, 75, 95, 115, or 135 mcg/day can be used to classify dietary cohorts.

In one embodiment, the tailored dietary program is presented as a diet plan for an individual; infant, child, teen, adolescent, adult, mature, senior; 0-1, 1-3, 2-5, 4-8, 7-12, 13-15, 14-20, 18-30, 25-45, 40-50, 45-60, 60-70, 70+ years of age, male or female.

In one embodiment, the tailored dietary program is prepared according to climatic condition and ambient temperature range. Temperature ranges may be classified as hot (90°-135°), warm) (70°-99°), cool (50°-75°), cold (33°-55°, below freezing (0°-37°), arctic (−50°-5°), or polar (−100°-−45°).

In one aspect, the tailored dietary program is manifested in packages, kits or modular food components that are used to complement the diet of the cohort or replace the caloric intake of the subject. The daily consumption of food according to plan may vary, but over a period of 1, 2, 4, 6, 8, 12, or more weeks, or as a lifestyle change, the tailored plan according to the invention provides at least 25, 50, 60, 70, 80, 90 or 100% of the total caloric intake of an individual.

Although it is important to deliver balanced complete diets balanced with respect to lipids, antioxidants, phytochemicals, vitamins, minerals, carbohydrates, microorganisms, fiber, and proteins, the perishability of certain essential fresh products may raise difficulties. However, some pre-formulated products such as lipids, nuts, seeds, dry herbs or herbal extracts, grains, and legumes have greater shelf life, and are less cumbersome to tailor. Some vegetable, fruit, meat, and seafood packs can also be developed, which require similar processing facilities and storage, i.e. produce/meat/seafood processing facilities with refrigeration. In addition to manufacturing and production advantages and industrial utility, this approach also retains a level of flexibility and gratification for the consumer in selecting the main dietary components.

Nutrients are selected from foods such as vegetables, fruits, grains, legumes (including lentils, peas, beans), herbs, spices, teas, cocoa, coffee, sweeteners, nuts, seeds, and oils; grains are selected from wheat, rice, corn, barley, spelt, oats, rye, buckwheat, millet, and quinoa; the vegetables are selected from asparagus, bell peppers, cucumber, eggplant, green beans, green peas, kale, romaine, spinach, squash summer and winter, tomato, carrots, romaine lettuce, radish, bitter gourd, okra, fenugreek leaves, broccoli, brussels sprout, cabbage, chard, cauliflower, mustard greens, collard greens, turnip greens, turnip, beets, potatoes, fungi, yams and sweet potatoes; the fruits are selected from apple, apricot, orange, pear, plum, banana, cantaloupe, grapes, grapefruit, papaya, mango, pineapple, blueberries, cranberries, figs, kiwi, prune, raspberries, pomegranate, strawberries and watermelon; the legumes are selected from black beans, dried peas, mung beans, garbanzo, kidney beans, lentils, lima beans, navy beans, pinto beans, pigeon peas, and soybeans; the herbs or spices are selected from asafetida, basil, bishop's weed, black pepper, cayenne pepper, chili pepper, cinnamon, cloves, coriander, cumin, dill, ginger, mustards seeds, oregano, peppermint, rosemary, sage, thyme, turmeric, fennel, garlic, onion, leeks, parsley, celery, cardamom, saffron, lime, lemon, tamarind, and mint, and the sweeteners are selected from molasses, cane juice, honey, maple syrup, dates, raisins, dried berries, figs, and sugar.

In some embodiments, the nutrients from the foods are extracted, and incorporated in a nutritional formulation in liquid, dry powder, or in topical cream or patch. Thus, formulations that provide the micronutrients to complement the remaining diet, may be oral compositions or topical in some embodiments.

In one aspect, the disclosure provides compositions that include seeds, nuts, and/or oils. In one embodiment the composition can include one or more edible oils, culinary nuts and/or seeds in their whole form or their oils such as, but not limited to acai oil, amaranth oil, apple seed oil, apricot kernel oil, argan oil, artichoke oil, avocado oil, babassu oil, ben oil, blackcurrant seed oil, borage seed oil, borneo tallow nut oil, bottle gourd oil, buffalo gourd oil, butter oil (anhydrous), canola oil (rapeseed), cape chestnut oil, carob pod oil, cocklebur oil, cocoa butter oil, cohune oil, coriander seed oil, corn oil, cottonseed oil, dika oil, evening primrose oil, false flax oil (camelina sativa), fish oil (cod liver), fish oil (herring), fish oil (menhaden), fish oil (salmon), fish oil (sardine), grapeseed oil, household lard, kapok seed oil, lallemantia oil, manila oil, meadowfoam seed oil, mustard oil, nutmeg butter, okra seed oil, palm oil, papaya seed oil, pequi oil, perilla oil, prune kernel oil, quinoa oil, ramtil oil, rice bran oil, royle oil, sacha inchi oil, safflower oil, sheanut oil, soybean lecithin oil, tea oil, thistle oil, tomato seed oil, ucuhuba butter oil, wheat germ oil, acorns, almonds, beech nuts, brazilnuts, breadnuts, candlenuts, chestnuts, chilacayote nuts, chilean hazel nuts, coconuts, cashews, colocynth nuts, filberts, hazelnut, hickory, kola nut, macadamia, mamoncillo, melon seeds, mongongo, obongo nut, olives, peanuts, pecans, pili nuts, pine nuts, pistachios, soya nuts, poppy seeds, pumpkin seeds, hemp seeds, flax seeds, sesame seeds, sunflower seeds, walnuts, and watermelon seeds.

In some embodiment, general formulations use sensitive/nutrient rich food items sparingly and with concern for adverse interactions: all nuts and seeds; oils and butters; eggs; some fruits: berries, papaya, apricot, fig, kiwi, pineapple; some vegetables: beets, yams, mustard greens, avocados; some legumes: soybeans and their products, pink lentils; some grains: barley, millet, buckwheat, oats; fungi (all kinds of mushrooms and yeasts); microorganisms (all kinds of probiotics, in excess they can cause problems, e.g. digestive issues and they can modulate metabolism of many foods); seafood including sea vegetables; herbs and spices in general: cinnamon, cloves, sage, turmeric; sweeteners: cane juice, honey, maple syrup; generally food folate, polyphenols, phytosterols, vitamin A, E, Se and fat containing foods. Ortolani C, Pastorello EA. Food allergies and food intolerances. Best Pract Res Clin Gastroenterol. 2006; 20:467-483. Lessof M H. Food intolerance and allergy—a review. Q J. Med. 1983; 52:111-119.

Typical interactions that are monitored comprise: seafood with nuts and seeds; seafood with phytochemicals; nuts and seeds, with berries, avocados, kiwi, papaya.

In one aspect, the total daily nutrients from all foods are within the ratios and ranges described herein and the compositions described herein are administered to an individual that falls within the age and calorie intake range as recommended. In a related embodiment, a 1-day, a 1-week, a 2-week, or a 1-month or more diet formulation or plan is provided.

In some embodiments, the nutritional formulations and diet plans are designed such that they provide greater (or less) than 10%, 15%, 20%, 25%, 30%, 35%, 40%, 45%, 50%, calories from protein, greater (or less) than 15%, 20%, 25%, 30%, 35%, 40%, 45%, 50% calories from lipids, and greater (or less) than 35%, 45%, 55%, 65%, 75%, 85% calories from carbohydrates.

In another aspect the diet comprises proteins, which proteins are from one or more of but not limited to legumes, eggs, cheese, milk, yogurt, poultry, seafood, and meat.

In some embodiments, carbohydrates are from greater (or less) than 40%, 50%, 70% intake of grains in calories, greater (or less) than 20%, 30%, 40% intake of vegetables in calories, and greater (or less) than 20%, 30%, 40% intake of fruits in calories. In a related aspect the calories from carbohydrates are additionally from one or more of spices, sweeteners, and beverages. In a further aspect the carbohydrates from grains are supplied by one or more of wheat, rice, corn, barley, spelt, oats, rye, buckwheat, millet, quinoa, and other grains.

In some embodiments, polyphenols, folate, phytosterols, alpha carotene, beta carotene, beta cryptoxanthin, betaine, choline, lycopene, and lutein/zeaxanthin are included in the formulations. For example, one or more polyphenols greater (or less) than 5, 10, 15, 20, 45, 70, 95, 115, 140, 165, 200, or 300 mg/day; and/or folate greater (or less) than 100, 200, 300, 400, 500, 600, 700, 800, 900, or 1000 mcg/day; and/or one or more phytosterols greater (or less) than 150, 200, 250, 300, 350, 450, 550, 650, 750, 850, or 1000 mg/day; and/or one or more carotenoid greater (or less) than 100, 300, 500, 1000, 3000, 5000, 8000, 10000, 12000, or 14000 mcg/day; and/or betaine and/or choline greater (or less) than 25, 50, 100, 200, 400, 500, or 600 mg/day. In some embodiments, these ranges represent limits for certain cohorts.

In some embodiments, antioxidants, and vitamins and minerals, e.g. Se are included in the formulations. For example, antioxidants greater (or less) than 25, 50, 100, 200, 400, 500, 600, or 1000 mg per day, or 1, 2, 4, 6, 8, or 10 g per day; and/or Se greater (or less) than 5, 10, 15, 20, 35, 55, 75, 95, 115, or 135 mcg/day can be used.

In some embodiments, one or more fibers are included in the formulations. For example, greater (or less) than 1, 5, 10, 20, 30, 40, 50 g per day.

Omega-6 to Omega-3 fatty acid ratios, depending on the cohort range from 1:1-50:1. In certain embodiments, the ratio is greater (or less) than 1:1, 5:1, 6:1, 7:1, 8:1, 10:1, 15:1, 20:1, 25:1, 30:1, 40:1, or 50:1.

Omega-9 to Omega-6 fatty acid ratios, depending on the cohort, range from 0.5:1-6:1. In various embodiments, the ratio is greater (or less) than 0.5:1, 1:1, 2:1, 3:1, 4:1, 5:1, or 6:1.

Total Fatty Acids to Monounsaturated fatty acid ratios, depending on the cohort, range from 1:1-15:1. In various embodiments, the ratio is greater (or less) than 1:1-2:1, 2:1-4:1, 4:1-6:1, 6:1-8:1, 8:1-10:1, 10-1:12:1, 12:1-15:1.

Monounsaturated to Polyunsaturated fatty acid ratios, depending on the cohort, range from 0.25:1-6:1. In various embodiments, the ratio is greater (or less) than 0.25:1, 1:1, 2:1, 3:1, 4:1, 5:1, or 6:1.

Monounsaturated to Saturated fatty acid ratios, depending on the cohort, range from 0.25:1-7:1. In various embodiments, the ratio is greater (or less) than 0.25:1, 1:1, 1.5:1, 2:1, 3:1, 5:1, 6:1 or 7:1.

Total Fatty Acids to Polyunsaturated fatty acid ratios, depending on the cohort, range from 1:1-15:1. In various embodiments, the ratio is greater (or less) than 1:1, 2:1, 3:1, 5:1, 7:1, 10:1, 12:1 or 15:1.

Total Fatty Acids to Saturated fatty acid ratios, depending on cohort, range from 1:1-15:1. In various embodiments, the ratio is greater (or less) than 1:1, 2:1, 3:1, 5:1, 7:1, 10:1, 12:1 or 15:1.

In some embodiments, the diet formulation calls for specific percentages of omega-9, omega-6, and omega-3 fatty acids within the following ranges (w/w, w/v, or v/v of total lipids). Omega-9 fatty acid, depending on the cohort, ranges from 10-90%. In some embodiments, omega-9 fatty acids comprise more (or less) than 10%, 15%, 20%, 25%, 30%, 35%, 40%, 45%, 50%, 55%, 60%, 65%, 70%, 80% or 90% of total lipids.

Omega-6 fatty acid, depending on the cohort, ranges from 4-75%. In some embodiments, omega-6 fatty acids comprise more (or less) than 5%, 10%, 15%, 20%, 25%, 30%, 35%, 40%, 45%, 50%, 55%, 60%, 65%, or 70% of total lipids.

Omega-3 fatty acid, depending on the cohort, ranges from 0.1-30%. In some embodiments, omega-3 fatty acids comprise more (or less) than 0.25%, 0.5%, 1%, 5%, 10%, 15%, 20%, 25% of total lipids. In some embodiments, from about 25% to about 100% by weight of the omega-3 fatty acids are long chain co-3 fatty acids. In some embodiments, from about 50% to about 100% by weight of the omega-3 fatty acids are long chain omega-3 fatty acids. For example, from about 60% to about 80%, or from about 70% to about 90%.

Vitamin E-alpha/gamma, depending on the cohort, ranges from 0.001-0.5%. In some embodiments, vitamin E-alpha/gamma comprise more (or less) than 0.01%, 0.05%, 0.10%, 0.15%, 0.20%, 0.25% or 0.30% of total lipids.

In some embodiments the average daily amount of omega-6 fatty acid according to the nutritional program ranges between 1-40 g. In embodiments, the daily amount of omega-6 fatty acid is more (or less) than 1, 2, 5, 10, 15, 20, 25, 30, 35, or 40 g.

In some embodiments the average daily amount of omega-3 fatty acid according to the nutritional program ranges between 0.1-15 g. In some embodiments, the daily amount of omega-3 fatty acid is more (or less) than 0.1, 0.2, 0.5, 1, 2, 5, 7, 10, 12, or 15 g.

In some embodiments, method of selection of foods for the seafood cohort comprises following steps: a) formulate a dietary protein component utilizing seafood, and meats, b) compute the range of micronutrients, proteins and lipids contained, c) select legumes, and grains to obtain additional proteins and desired carbohydrates, d) select vegetables and fruits to meet micronutrient requirements, e) select probiotics and prebiotics, and f) select oils and other foods to meet remaining lipid, protein, phytochemical, antioxidants and vitamins and minerals requirements. In some embodiments, for a seafood based cohort hulled grains may be preferred, for example, white rice may be used instead of brown rice, a less intense variety of wheat may be used, and lesser than 10% of grain calories on the average may be used from spelt, barley, oats, rye, buckwheat, millet and quinoa. Similarly, use of mustard greens, yams, fungi, winter squash, and berries may be restricted because of their nutrients density and interaction potential with nutrients in seafood. In a related aspect, preferable ratio of omega-6 to omega-3 may be, 2:1-4:1, 4:1-10:1, 10:1-15:1. In a related aspect, seafood cohorts may consume relatively high amounts of one or more of long-chain omega-3, and Se, which may limit tolerance for certain phytochemicals, for example flavonoids and folate. In some embodiments, diets high in long-chain omega-3 and/or Se are complemented with low phytochemicals, particularly flavonoids, and folate.

In some embodiments, method of selection of foods for the meat (red) based cohort comprises following steps: a) formulate a dietary protein component utilizing meats, b) compute the range of micronutrients, proteins and lipids contained, c) select legumes and grains to obtain additional proteins and desired carbohydrates, d) select vegetables and fruits to add micronutrients, e) select probiotics and prebiotics, and f) select herbs, nuts, seeds, and oils to meet remaining lipid, phytochemical particularly flavonoids and sterols, antioxidants and vitamins and minerals requirements. In some embodiments, for a meat based cohort whole grains are utilized preferentially. Similarly, mustard greens, yams, fungi, winter, squash, and berries may be utilized preferentially because of their nutrient density. In a related aspect, preferable ratio of omega-6 to omega-3 may be 0.5:1-4:1, 4:1-10:1. In a related aspect, meat cohorts may consume relatively high amounts of one or more of long-chain omega-6, and certain saturated fatty acids, and their diet may be inherently low in certain phytochemicals, for example flavonoids and folate. In some embodiments, diets high in long-chain omega-6 are complemented with high antioxidants, such as Vitamin E, and phytochemicals, particularly flavonoids, and folate.

In some embodiments, method of selection of foods for the plant based ovo-lacto vegetarian cohort comprises following steps: a) formulate a dietary protein component utilizing legumes and dairy products, b) compute the range of micronutrients, proteins, and lipids contained, c) select grains to obtain desired carbohydrate and proteins, d) select vegetables and fruits to add micronutrients, e) select probiotics and prebiotics, and f) select herbs, nuts, seeds, and oils to meet remaining lipid, phytochemical, antioxidants and vitamins and minerals requirements. In some embodiments, for a plant based cohort some of the grains are hulled, for example, white rice may be used instead of brown rice, a less intense variety of wheat may be used, and lesser than 15% of grain calories on the average may be used from spelt, barley, oats, rye, buckwheat, millet and quinoa. Similarly, use of mustard greens, yams, fungi, winter, squash, and berries may be restricted because of their nutrients density and interaction potential with nutrients in legumes. In a related aspect, preferable ratio of omega-6 to omega-3 may be, 4:1-10:1, 10:1-15:1, 15:1-20:1. In a related aspect, ovo-lacto cohorts may consume relatively high amounts of one or more polyphenols, particularly isoflavones, which may limit tolerance for certain other phytochemicals, for example folate and/or phytochemicals found in whole grains. In some embodiments, diets high in legumes are complemented with low phytochemicals, from whole grains.

In some aspects, the nutritional/diet plan or food compositions developed therefrom can serve as a medicaments or compositions for use in prophylaxis or treatment of certain diseases or medical conditions. Medicaments can be based on a subject's dietary habits around typical consumption of phytochemicals, antioxidants, and other nutrients. Appropriate supplements, medications or pharmaceutical drugs can also be administered to/by such dietary cohorts because their requirements, biochemistry, and gene expression may be influenced in a certain predictable way.

In some aspects, the nutritional/diet plan or food compositions developed therefrom can serve as a medicaments or compositions for use in prophylaxis or alleviation of one or more symptoms associated with a disease or condition selected from: menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases.

Potential benefits of the tailored dietary or nutritional programs include: a) lipid, antioxidant, phytochemical, vitamin and mineral delivery within the optimal range considering main food preferences, age, gender, size, medical condition, family history, and climatic temperature; b) synergistic use of different natural sources to deliver an array of nutrients; c) reduction of some potential harmful interactions; d) managed expression of oxidation products; e) optimization of gene expression; f) steady delivery of lipids and phytochemicals to stabilize immunity and physiology; g) satiety and its perception, since nutrient requirements are balanced; and h) caloric restriction.

In one aspect, the invention relates to a computer program storage device readable by a machine or processor and containing a set of instructions which, when read by the machine, causes execution of a bioinformatics method for generating a compilation of dietary ingredients comprising a nutritional plan. In some embodiments, the method may be stored on a computer-readable medium having computer-executable instructions for performing the method. A computer-readable medium may include, but is not limited to, a compact disc (CD), a USB thumb drive, an optical drive, or a magnetic drive. Other types of computer-readable media may be used as well, such as those presently known in the art and those yet to be discovered. The method is executed on a computational device comprising a processor, at least one memory and optionally, a display and a measuring device. Stored in the memory are parameters that allow classification of an individual's dietary pattern input into the memory into at least one predetermined dietary cohort by the processor. Also stored in the memory are modules of nutritional plans that have been developed as appropriate for each dietary cohort. In some aspects, the device is connectable to other devices by wired or wireless connection or over LAN or WAN. The computer program operates in response to user inputs, which in some embodiments include dietary habits for an individual (e.g., approximate daily consumption values in step 1 of Table 4). User inputs may be remote, via web connection. The computer program is configured to identify dietary cohort and/or calculate ranges in step 2 of Table 4, and develop complementing nutritional supplements in accordance with this disclosure, and such nutritional supplements may be based upon culinary preferences for the individual, which may also be input into the system.

As defined herein, a therapeutically effective amount of the nutrient (i.e., an effective amount) may range from about 0.0001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

According to another aspect, one or more kits of parts can be envisioned by the person skilled in the art, the kits of parts to perform at least one of the methods herein disclosed, the kit of parts comprising two or more compositions, the compositions comprising alone or in combination an effective amount of the compositions disclosed herein according to the at least one of the above mentioned methods.

The kits possibly include also compositions/formulations comprising active agents, identifiers of a biological event, or other compounds identifiable by a person skilled upon reading of the present disclosure. The kit can also comprise at least one composition comprising an effective amount of the compositions disclosed herein or a cell line. The compositions and the cell line of the kits of parts to be used to perform the at least one method herein disclosed according to procedure identifiable by a person skilled in the art.

According to one aspect, complementary modules or packages are provided that suit a particular diet plan. Such modules or packages may be embodied as vegetable/vegetable juice packs, fruit/fruit juice packs, dry grain packs, cereal packs, legume/grain/nuts and/or seeds packs, meat/seafood packs, herbs, lipids, desserts, milks, yogurts and the like, or a combination thereof in cooked, uncooked, processed or unprocessed form.

Each module is marked or is otherwise associated with indication that it is suitable for consumption by an individual with a specific dietary profile or a dietary cohort or further sub-sections thereof based on additional factors such as gender, age, location, climate, medical condition and the like. Consumption of a suitable module ensures an optimized pattern of nutrient profile in the consumer, in particular nutrients that are sensitive to narrow fluctuations.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Diet Formulations

In one embodiment, a diet plan is provided which includes the 25%-45% of calories from fat, which are supplied by the lipid compositions described herein. In an exemplary general diet plan, macronutrients provide 35-65% of calories from carbohydrates, 10-45% of calories from proteins and 15-45% of calories from lipids. The general diet formulation comprises one or more of the components listed in Table 5 below, wherein the upper limits are set on the basis of levels of the micronutrients present in the each food item and the sensitivity of the food item. Thus, nutritional formulations are provided to the individual to balance the individual's diet within the following ranges shown in Table 5, with listed ingredients being alternatives for use individually or together (e.g., in one or more nutritional formulation disclosed herein).

In one aspect, one or more of the food items are provided in individual modules or packages of food or drink. In one aspect, each package comprises a label indicating its suitability for consumption according to a general diet plan and optionally, maximum amounts for average daily consumption to maintain a health benefit according to the invention.

TABLE 5
General Diet Formulation

| | | | | Upper | |
|---|---|---|---|---|---|
| | | | | limit of | |
| | | | | Avg. Daily | Upper limit |
| | | | | Amounts | of Avg. |
| | | 50-70% of | | (uncooked, | Daily |
| | | carbohydrate | | Grams/ | Servings |
| Grains (one or more of) | | calories | | Calories) | (cups) |
| 1 | Wheat | <50% | of grains | 114 | 3.5 |
| 2 | Rice | <50% | of grains | 114 | 2.5 |
| 3 | Corn | <20% | of grains | 46 | 1 |
| 4 | Barley | <20% | of grains | 46 | 1 |
| 5 | Spelt | <20% | of grains | 46 | 1 |
| 6 | Oats | <20% | of grains | 46 | 1 |
| 7 | Rye | <20% | of grains | 46 | 1 |
| 8 | Buckwheat | <15% | of grains | 34 | 0.75 |
| 9 | Millet | <15% | of grains | 34 | 0.75 |
| 10 | Quinoa | <15% | of grains | 34 | 0.75 |
| 11 | Other Grains | <10% | of grains | 23 | 0.5 |
| Vegetables (one or more of) | | 15-40% of carbohydrate calories | | | |
| 1 | Bell Peppers, Cucumber, Eggplant, | <50% | of | | 5 |
| | Green beans, Green peas, Spinach, | | vegetables | | |
| | Squash summer, Tomato, Okra, | | | | |
| | Potatoes | | | | |
| 2 | Asparagus, Broccoli, Brussels Sprout, | <40% | of | | 4 |
| | Cabbage, Carrots, Chard, Cauliflower, | | vegetables | | |
| | Kale, Collard Greens, Fenugreek | | | | |
| | Leaves, Romaine Lettuce | | | | |
| 3 | Turnip, Turnip Greens, Beets, Yams, | <35% | of | | 2 |
| | Sweet Potatoes, Winter squash, Bitter | | vegetables | | |
| | Gourd, Radish, Mustard Greens | | | | |
| 4 | Fungi includes all mushrooms | <25% | of | | 1 |
| | | | vegetables | | |
| 5 | Other Vegetables | <15% | of | | 0.5 |
| | | | vegetables | | |
| Fruits (one or more of) | | 10-30% of carbohydrate calories | | | |
| 1 | Apple, Orange, Pear, Banana, | <75% | of fruits | | 2 |
| | Cantaloupe, Grapes | | | | |
| 2 | Apricots, Grapefruit, Papaya, Mango, | <50% | of fruits | | 1 |
| | Pineapple | | | | |
| 3 | Blueberries, Cranberries, Figs, Kiwi, | <35% | of fruits | | 1 |
| | Prune, Raspberries, Pomegranate, | | | | |
| | Strawberries, Watermelon, Plum | | | | |
| 4 | Other fruits | <15% | of fruits | | 0.5 |
| Spices (one or more of) | | <7% of carbohydrate calories | | | |
| | Basil, Black pepper, Cayenne pepper, | | | | 3 tsp. |
| | Chili Pepper, Cinnamon, Cloves, | | | | |
| | Coriander seeds and leaves, Cumin, | | | | |
| | Dill, Ginger, Mustard Seeds, Oregano, | | | | |
| | Peppermint leaves, Rosemary, Sage, | | | | |
| | Thyme, Turmeric, Fennel, Garlic, | | | | |
| | Onion, Leeks, Parsley, Celery, | | | | |
| | Cardamom, Saffron, Lime, Lemon, | | | | |
| | Tamarind, Mint, Vinegar, other | | | | |
| Sweeteners (one or more of) | | <7% of carbohydrate calories | | | |
| | Molasses, Cane Juice, Honey, Maple | | | | 2 tbs. |
| | Syrup, Dates, Raisins, Dried Berries, | | | | |
| | Figs, Sugar, other | | | | |
| Beverages (one or more of) | | <5% of carbohydrate calories | | | |
| | Green tea, Black tea, cocoa, coffee, | | | | 3 |
| | alcohol, other | | | | |
| Proteins (one or more of) | | 10-45% of calories | | | |
| | Legumes: Black beans, Dried Peas, | <75% | of protein | 675 | 3 |
| | Mung beans, Garbanzo, Kidney beans, | | calories | | |
| | Lentils, Lima beans, Navy beans, Pinto | | | | |
| | beans, Soybeans | | | | |
| | Meat | <60% | of protein | 540 | 2 |
| | | | calories | | |
| | Poultry | <60% | of protein | 540 | 2 |
| | | | calories | | |
| | Seafood | <50% | of protein | 450 | 1 |
| | | | calories | | |
| | Milk | <35% | of protein | 315 | 2 |
| | | | calories | | |
| | Cheese | <20% | of protein | 180 | 1 |
| | | | calories | | |
| | Eggs | <15% | of protein | 135 | 1 |
| | | | calories | | |
| | Yogurt | <15% | of protein | 135 | 1 |
| | | | calories | | |
| | Other | <15% | of protein | 135 | 1 |
| | | | calories | | |
| Lipids (one or more of) | | 15-45% of calories | | | |
| | Peanut oil, olive oil, sunflower oil, | <75% | of lipid | 675 | |
| | safflower oil, corn oil | | calories | | |
| | Coconut Oil, Butter or butter oil | <45% | of lipid | 405 | |
| | | | calories | | |
| | Olives, Walnuts, flaxseeds | <45% | of lipid | 405 | |
| | | | calories | | |
| | Almonds, cashews, pistachios, peanuts | <30% | of lipid | 270 | |
| | | | calories | | |
| | Sesame seeds, flaxseeds, pumpkin | <25% | of lipid | 225 | |
| | seeds, sunflower seeds | | calories | | |
| | Other | <10% | of lipid | 100 | |
| | | | calories | | |

| Lipid Ratios: | | |
|---|---|---|
| | Omega-6:omega-3 | 0.5:1-20:1 |
| | Omega-9:omega-6 | 1:1-3:1 |
| | Mono:Poly | 1:1-3:1 |
| | Mono:Sat | 1:1-5:1 |
| Omega fatty acids: | | |
| | Omega-6 | 1-40 g |
| | Omega-3 | 0.1-20 g |

### Example 2

### Diet Formulation for Cohort: Seafood

In one embodiment, a diet plan is provided for a cohort who derives 2%-40% of calories from seafood per day/week/month. Such individuals can generally be classified as seafood-heavy.

A 1-day, a 1-week, a 2-week, or a 1-month diet plan is provided which includes the 2%-40% of calories from seafood, and the remaining 60%-98% of calories are supplied by a diet including the following components, ranges specified in calories. The components in Table 6 are selected such that levels of sensitive nutrients are optimized. Thus, nutritional formulations are provided to the individual to balance the individual's diet within the following ranges shown in Table 6, with listed ingredients being alternatives for use individually or together (e.g., in one or more nutritional formulations described herein).

The seafood cohort diet formulation comprises one or more of the components listed in Table 6 below, wherein the upper limits are set on the basis of levels of the micronutrients present in the each food item, and the sensitivity of the food item. In one aspect, one or more of the food items are provided in individual modules or packages of food or drink. In one aspect, each package comprises a label indicating its suitability for consumption according to a seafood cohort diet plan and optionally, maximum amounts for average daily consumption to maintain a health benefit according to the invention.

TABLE 6
Seafood Cohort Diet Formulation

| | | | | Upper | |
|---|---|---|---|---|---|
| | | | | limit of | |
| | | | | Avg. Daily | Upper limit |
| | | | | Amounts | of Avg. |
| | | 50-70% of | | (uncooked, | Daily |
| Grains (one or more of) | | carbohydrate | | Grams/ | Servings |
| (hulled grains preferred) | | calories | | Calories) | (cups) |
| 1 | Wheat | <50% | of grains | 114 | 3.5 |
| 2 | Rice | <50% | of grains | 114 | 2.5 |
| 3 | Corn | <10% | of grains | 23 | 0.5 |
| 5 | Spelt | <10% | of grains | 23 | 0.5 |
| 6 | Oats | <10% | of grains | 23 | 0.5 |
| 10 | Quinoa | <10% | of grains | 15 | 0.5 |
| 11 | Other Grains | <10% | of grains | 23 | 0.5 |
| Vegetables (one or more of) | | 15-40% of carbohydrate calories | | | |
| 1 | Bell Peppers, Cucumber, Eggplant, | <50% | of | | 5 |
| | Green beans, Green peas, Spinach, | | vegetables | | |
| | Squash summer, Tomato, Okra, | | | | |
| | Potatoes | | | | |
| 2 | Asparagus, Broccoli, Brussels Sprout, | <30% | of | | 3 |
| | Cabbage, Carrots, Chard, Cauliflower, | | vegetables | | |
| | Kale, Collard Greens, Fenugreek | | | | |
| | Leaves, Romaine Lettuce | | | | |
| 3 | Turnip, Turnip Greens, Beets, Yams, | <10% | of | | 0.5 |
| | Sweet Potatoes, Winter squash, Bitter | | vegetables | | |
| | Gourd, Radish, Mustard Greens | | | | |
| 4 | Fungi includes all mushrooms | <10% | of | | 0.5 |
| | | | vegetables | | |
| 5 | Other Vegetables | <15% | of | | 0.5 |
| | | | vegetables | | |
| Fruits (one or more of) | | 10-30% of carbohydrate calories | | | |
| 1 | Apple, Orange, Pear, Banana, | <75% | of fruits | | 2 |
| | Cantaloupe, Grapes | | | | |
| 2 | Apricots, Grapefruit, Papaya, Mango, | <30% | of fruits | | 0.5 |
| | Pineapple | | | | |
| 3 | Blueberries, Cranberries, Figs, Kiwi, | <15% | of fruits | | 0.25 |
| | Prune, Raspberries, Pomegranate, | | | | |
| | Strawberries, Watermelon, Plum | | | | |
| 4 | Other fruits | <15% | of fruits | | 0.25 |
| Spices (one or more of) | | <5% of carbohydrate calories | | | |
| | Basil, Black pepper, Cayenne pepper, | | | | 2 tsp. |
| | Chili Pepper, Cinnamon, Cloves, | | | | |
| | Coriander seeds and leaves, Cumin, | | | | |
| | Dill, Ginger, Mustard Seeds, Oregano, | | | | |
| | Peppermint leaves, Rosemary, Sage, | | | | |
| | Thyme, Turmeric, Fennel, Garlic, | | | | |
| | Onion, Leeks, Parsley, Celery, | | | | |
| | Cardamom, Saffron, Lime, Lemon, | | | | |
| | Tamarind, Mint, Vinegar, other | | | | |
| Sweeteners (one or more of) | | <7% of carbohydrate calories | | | |
| | Molasses, Cane Juice, Honey, Maple | | | | 2 tbs. |
| | Syrup, Dates, Raisins, Dried Berries, | | | | |
| | Figs, Sugar, other | | | | |
| Beverages (one or more of) | | <5% of carbohydrate calories | | | |
| | Green tea, Black tea, cocoa, coffee, | | | | 3 |
| | alcohol, other | | | | |
| Proteins (one or more of) | | 10-45% of calories | | | |
| | Legumes: Black beans, Dried Peas, | <50% | of protein | 450 | 2 |
| | Mung beans, Garbanzo, Kidney beans, | | calories | | |
| | Lentils, Lima beans, Navy beans, Pinto | | | | |
| | beans, Soybeans | | | | |
| | Meat | <60% | of protein | 540 | 2 |
| | | | calories | | |
| | Poultry | <60% | of protein | 540 | 2 |
| | | | calories | | |
| | Seafood | <50% | of protein | 450 | 1 |
| | | | calories | | |
| | Milk | <35% | of protein | 315 | 2 |
| | | | calories | | |
| | Cheese | <20% | of protein | 180 | 1 |
| | | | calories | | |
| | Eggs | <15% | of protein | 135 | 1 |
| | | | calories | | |
| | Yogurt | <10% | of protein | 135 | 1 |
| | | | calories | | |
| | Other | <15% | of protein | 135 | 1 |
| | | | calories | | |
| Lipids (one or more of) | | 15-45% of calories | | | |
| | Peanut oil, olive oil, sunflower oil, | <75% | of lipid | 675 | |
| | safflower oil, corn oil | | calories | | |
| | Coconut Oil, Butter or butter oil | <45% | of lipid | 405 | |
| | | | calories | | |
| | Other | <10% | of lipid | 100 | |
| | | | calories | | |

| Lipid Ratios: | | |
|---|---|---|
| | Omega-6:omega-3 | 2:1-15:1 |
| | Omega-9:omega-6 | 1:1-3:1 |
| | Mono:Poly | 1:1-3:1 |
| | Mono:Sat | 1:1-5:1 |
| Omega fatty acids: | | |
| | Omega-6 | 1-35 g |
| | Omega-3 | 0.1-20 g |

### Example 3

### Diet Formulation for Cohort: Meat

In one embodiment, a diet plan is provided for a cohort who derives 10%-50% of calories from meat per day/week/month. Such individuals can generally be classified as meat-heavy.

A 1-day, a 1-week, a 2-week, or a 1-month diet plan is provided which includes the 10%-50% of calories from meat, and the remaining 50%-90% of calories are supplied by a diet including the following components, ranges specified in calories. Thus, nutritional formulations are provided to the individual to balance the individual's diet within the following ranges shown in Table 7, with listed ingredients being alternatives for use individually or together (e.g., with one or more nutritional formulations described herein).

The components in Table 7 are selected such that levels of sensitive nutrients are optimized.

The meat cohort diet formulation comprises one or more of the components listed in Table 7 below, wherein the upper limits are set on the basis of levels of the micronutrients present in the each food item, and the sensitivity of the food item. In one aspect, one or more of the food items are provided in individual modules or packages of food or drink. In one aspect, each package comprises a label indicating its suitability for consumption according to a meat cohort diet plan and optionally, maximum amounts for average daily consumption to maintain a health benefit according to the invention.

TABLE 7
Meat Cohort Diet Formulation

| | | | | Upper | |
|---|---|---|---|---|---|
| | | | | limit of | |
| | | | | Avg. Daily | Upper limit |
| | | | | Amounts | of Avg. |
| | | 50-70% of | | (uncooked | Daily |
| | | carbohydrate | | Grams/ | Servings |
| Grains (one or more of) | | calories | | Calories) | (cups) |
| 1 | Wheat | <50% | of grains | 114 | 3.5 |
| 2 | Rice | <50% | of grains | 114 | 2.5 |
| 3 | Corn | <20% | of grains | 46 | 1 |
| 4 | Barley | <20% | of grains | 46 | 1 |
| 5 | Spelt | <20% | of grains | 46 | 1 |
| 6 | Oats | <20% | of grains | 46 | 1 |
| 7 | Rye | <20% | of grains | 46 | 1 |
| 8 | Buckwheat | <15% | of grains | 34 | 0.75 |
| 9 | Millet | <15% | of grains | 34 | 0.75 |
| 10 | Quinoa | <15% | of grains | 34 | 0.75 |
| 11 | Other Grains | <10% | of grains | 23 | 0.5 |
| Vegetables (one or more of) | | 15-40% of carbohydrate calories | | | |
| 1 | Bell Peppers, Cucumber, Eggplant, | <50% | of | | 5 |
| | Green beans, Green peas, Spinach, | | vegetables | | |
| | Squash summer, Tomato, Okra, | | | | |
| | Potatoes | | | | |
| 2 | Asparagus, Broccoli, Brussels Sprout, | <40% | of | | 4 |
| | Cabbage, Carrots, Chard, Cauliflower, | | vegetables | | |
| | Kale, Collard Greens, Fenugreek | | | | |
| | Leaves, Romaine Lettuce | | | | |
| 3 | Turnip, Turnip Greens, Beets, Yams, | <35% | of | | 2 |
| | Sweet Potatoes, Winter squash, Bitter | | vegetables | | |
| | Gourd, Radish, Mustard Greens | | | | |
| 4 | Fungi includes all mushrooms | <25% | of | | 1 |
| | | | vegetables | | |
| 5 | Other Vegetables | <15% | of | | 0.5 |
| | | | vegetables | | |
| Fruits (one or more of) | | 10-30% of carbohydrate calories | | | |
| 1 | Apple, Orange, Pear, Banana, | <75% | of fruits | | 2 |
| | Cantaloupe, Grapes | | | | |
| 2 | Apricots, Grapefruit, Papaya, Mango, | <50% | of fruits | | 1 |
| | Pineapple | | | | |
| 3 | Blueberries, Cranberries, Figs, Kiwi, | <35% | of fruits | | 1 |
| | Prune, Raspberries, Pomegranate, | | | | |
| | Strawberries, Watermelon, Plum | | | | |
| 4 | Other fruits | <15% | of fruits | | 1 |
| Spices (one or more of) | | <7% of carbohydrate calories | | | |
| | Basil, Black pepper, Cayenne pepper, | | | | 3 tsp. |
| | Chili Pepper, Cinnamon, Cloves, | | | | |
| | Coriander seeds and leaves, Cumin, | | | | |
| | Dill, Ginger, Mustard Seeds, Oregano, | | | | |
| | Peppermint leaves, Rosemary, Sage, | | | | |
| | Thyme, Turmeric, Fennel, Garlic, | | | | |
| | Onion, Leeks, Parsley, Celery, | | | | |
| | Cardamom, Saffron, Lime, Lemon, | | | | |
| | Tamarind, Mint, Vinegar, other | | | | |
| Sweeteners (one or more of) | | <7% of carbohydrate calories | | | |
| | Molasses, Cane Juice, Honey, Maple | | | | 2 tbs. |
| | Syrup, Dates, Raisins, Dried Berries, | | | | |
| | Figs, Sugar, other | | | | |
| Beverages (one or more of) | | <5% of carbohydrate calories | | | |
| | Green tea, Black tea, cocoa, coffee, | | | | 3 |
| | alcohol, other | | | | |
| Proteins (one or more of) | | 10-45% of calories | | | |
| | Legumes: Black beans, Dried Peas, | <75% | of protein | 675 | 3 |
| | Mung beans, Garbanzo, Kidney beans, | | calories | | |
| | Lentils, Lima beans, Navy beans, Pinto | | | | |
| | beans, Soybeans | | | | |
| | Meat | <60% | of protein | 540 | 2 |
| | | | calories | | |
| | Poultry | <60% | of protein | 540 | 2 |
| | | | calories | | |
| | Seafood | <50% | of protein | 450 | 1 |
| | | | calories | | |
| | Milk | <35% | of protein | 315 | 2 |
| | | | calories | | |
| | Cheese | <20% | of protein | 180 | 1 |
| | | | calories | | |
| | Eggs | <15% | of protein | 135 | 1 |
| | | | calories | | |
| | Yogurt | <15% | of protein | 135 | 1 |
| | | | calories | | |
| | Other | <15% | of protein | 135 | 1 |
| | | | calories | | |
| Lipids (one or more of) | | 15-45% of calories | | | |
| | Peanut oil, olive oil, sunflower oil, | <50% | of lipid | 675 | |
| | safflower oil, corn oil | | calories | | |
| | Coconut Oil, Butter or butter oil | <45% | of lipid | 405 | |
| | | | calories | | |
| | Olives, Walnuts, flaxseeds | <50% | of lipid | 405 | |
| | | | calories | | |
| | Almonds, cashews, pistachios, peanuts | <30% | of lipid | 270 | |
| | | | calories | | |
| | Sesame seeds, flaxseeds, pumpkin | <25% | of lipid | 225 | |
| | seeds, sunflower seeds | | calories | | |
| | Other | <10% | of lipid | 100 | |
| | | | calories | | |

| Lipid Ratios: | | |
|---|---|---|
| | Omega-6:omega-3 | 0.5:1-10:1 |
| | Omega-9:omega-6 | 1:1-3:1 |
| | Mono:Poly | 1:1-3:1 |
| | Mono:Sat | 1:1-5:1 |
| Omega fatty acids: | | |
| | Omega-6 | 1-40 g |
| | Omega-3 | 0.1-20 g |

### Example 4

### Diet Formulation for Cohort: Legumes, Vegetables and Fruits

In one embodiment, a diet plan is provided for a cohort which derives 20%-80% of calories from legumes, vegetables and fruits per day/week/month. Such individuals are generally considered to have a vegetable heavy diet.

A 1-day, a 1-week, a 2-week, or a 1-month diet plan is provided which includes the 20%-80% of calories from legumes, vegetables and fruits, and the remaining 80%-20% of calories are supplied by a diet including the following components, ranges specified in calories. The components in Table 8 are selected such that levels of sensitive nutrients are optimized. Thus, nutritional formulations are provided to the individual to balance the individual's diet within the following ranges shown in Table 8, with listed ingredients being alternatives for use individually or together (e.g., in one or more nutritional formulations described herein).

The legumes, vegetables and fruits cohort diet formulation comprises one or more of the components listed in Table 8 below, wherein the upper limits are set on the basis of levels of the micronutrients present in the each food item, and the sensitivity of the food item. In one aspect, one or more of the food items are provided in individual modules or packages of food or drink. In one aspect, each package comprises a label indicating its suitability for consumption according to a legumes, vegetables and fruits cohort diet plan and optionally, maximum amounts for average daily consumption to maintain a health benefit according to the invention.

TABLE 8
Legumes, Vegetables and Fruits Cohort Diet Formulation

| | | | | Upper limit | |
|---|---|---|---|---|---|
| | | | | of Avg. | Upper limit |
| | | | | Daily | of Avg. |
| | | 50-70% of | | Amounts | Daily |
| Grains (one or more of) | | carbohydrate | | (Uncooked | Servings |
| (selectively hulled grains preferred) | | calories | | Grams/Cal) | (cups) |
| 1 | Wheat | <50% | of grains | 114 | 3.5 |
| 2 | Rice | <50% | of grains | 114 | 2.5 |
| 3 | Corn | <20% | of grains | 46 | 1 |
| 4 | Barley | <15% | of grains | 34 | 0.75 |
| 5 | Spelt | <15% | of grains | 34 | 0.75 |
| 6 | Oats | <15% | of grains | 34 | 0.75 |
| 7 | Rye | <15% | of grains | 34 | 0.75 |
| 8 | Buckwheat | <10% | of grains | 23 | 0.5 |
| 9 | Millet | <10% | of grains | 23 | 0.5 |
| 10 | Quinoa | <10% | of grains | 23 | 0.5 |
| 11 | Other Grains | <10% | of grains | 23 | 0.5 |
| Vegetables (one or more of) | | 15-40% of carbohydrate calories | | | |
| 1 | Bell Peppers, Cucumber, Eggplant, | <50% | of | | 5 |
| | Green beans, Green peas, Spinach, | | vegetables | | |
| | Squash summer, Tomato, Okra, | | | | |
| | Potatoes | | | | |
| 2 | Asparagus, Broccoli, Brussels Sprout, | <40% | of | | 4 |
| | Cabbage, Carrots, Chard, Cauliflower, | | vegetables | | |
| | Kale, Collard Greens, Fenugreek | | | | |
| | Leaves, Romaine Lettuce | | | | |
| 3 | Turnip, Turnip Greens, Beets, Yams, | <35% | of | | 2 |
| | Sweet Potatoes, Winter squash, Bitter | | vegetables | | |
| | Gourd, Radish, Mustard Greens | | | | |
| 4 | Fungi includes all mushrooms | <25% | of | | 1 |
| | | | vegetables | | |
| 5 | Other Vegetables | <15% | of | | 0.5 |
| | | | vegetables | | |
| Fruits (one or more of) | | 10-30% of carbohydrate calories | | | |
| 1 | Apple, Orange, Pear, Banana, | <75% | of fruits | | 2 |
| | Cantaloupe, Grapes | | | | |
| 2 | Apricots, Grapefruit, Papaya, Mango, | <25% | of fruits | | 0.5 |
| | Pineapple | | | | |
| 3 | Blueberries, Cranberries, Figs, Kiwi, | <25% | of fruits | | 0.5 |
| | Prune, Raspberries, Pomegranate, | | | | |
| | Strawberries, Watermelon, Plum | | | | |
| 4 | Other fruits | <15% | of fruits | | 5 |
| Spices (one or more of) | | <7% of carbohydrate calories | | | |
| | Basil, Black pepper, Cayenne pepper, | | | | 3 tsp. |
| | Chili Pepper, Cinnamon, Cloves, | | | | |
| | Coriander seeds and leaves, Cumin, | | | | |
| | Dill, Ginger, Mustard Seeds, Oregano, | | | | |
| | Peppermint leaves, Rosemary, Sage, | | | | |
| | Thyme, Turmeric, Fennel, Garlic, | | | | |
| | Onion, Leeks, Parsley, Celery, | | | | |
| | Cardamom, Saffron, Lime, Lemon, | | | | |
| | Tamarind, Mint, Vinegar, other | | | | |
| Sweeteners (one or more of) | | <7% of carbohydrate calories | | | |
| | Molasses, Cane Juice, Honey, Maple | | | | 2 tbs. |
| | Syrup, Dates, Raisins, Dried Berries, | | | | |
| | Figs, Sugar, other | | | | |
| Beverages (one or more of) | | <5% of carbohydrate calories | | | |
| | Green tea, Black tea, cocoa, coffee, | | | | 3 |
| | alcohol, other | | | | |
| Proteins (one or more of) | | 10-45% of calories | | | |
| | Legumes: Black beans, Dried Peas, | <75% | of protein | 675 | 3 |
| | Mung beans, Garbanzo, Kidney beans, | | calories | | |
| | Lentils, Lima beans, Navy beans, Pinto | | | | |
| | beans, Soybeans | | | | |
| | Milk | <35% | of protein | 315 | 2 |
| | | | calories | | |
| | Cheese | <20% | of protein | 180 | 1 |
| | | | calories | | |
| | Eggs | <15% | of protein | 135 | 1 |
| | | | calories | | |
| | Yogurt | <15% | of protein | 135 | 1 |
| | | | calories | | |
| | Other | <15% | of protein | 135 | 1 |
| | | | calories | | |
| Lipids (one or more of) | | 15-45% of calories | | | |
| | Peanut oil, olive oil, sunflower oil, | <75% | of lipid | 675 | |
| | safflower oil, corn oil | | calories | | |
| | Coconut Oil, Butter or butter oil | <45% | of lipid | 405 | |
| | | | calories | | |
| | Olives, Walnuts, flaxseeds | <45% | of lipid | 405 | |
| | | | calories | | |
| | Almonds, cashews, pistachios, peanuts | <30% | of lipid | 270 | |
| | | | calories | | |
| | Sesame seeds, flaxseeds, pumpkin | <25% | of lipid | 225 | |
| | seeds, sunflower seeds | | calories | | |
| | Other | <10% | of lipid | 100 | |
| | | | calories | | |

| Lipid Ratios: | | |
|---|---|---|
| | Omega-6:omega-3 | 4:1-20:1 |
| | Omega-9:omega-6 | 1:1-3:1 |
| | Mono:Poly | 1:1-3:1 |
| | Mono:Sat | 1:1-5:1 |
| Omega fatty acids: | | |
| | Omega-6 | 1-40 g |
| | Omega-3 | 0.1-15 g |

### Example 5

### Packaging and Labeling of a Dietary Module

In one embodiment, dietary module according to a diet plan for a specific cohort or dietary profile comprises vegetable/vegetable juice packs, fruit/fruit juice packs, dry grain packs, cereal packs, legume/grain/nuts and/or seeds packs, meat or seafood packs, herbs, lipids, desserts, milks, yogurts and the like, or a combination thereof. The appropriate cohort or dietary profile for who the package is designed is indicated in association with the package. Each package is designed to provide less than 25% of calories per day/week/month which is indicated in association with the package.

Other nutritional information optionally indicated in association with package comprises information about ingredients, consumption limits, list of nutrients, and the like.

### Example 6

### Case Study on Hypercholesterolemia, Cardiovascular Disease

The host subject experienced hypercholesterolemia on a vegetarian diet low in fat, mostly olive oil (75% monounsaturated fat), a daily fish oil supplement of 1 gram, and a daily total essential fatty acids (EFA) supplement of 1 gram. As part of the treatment, the fish oil and EFA supplements were discontinued. The subject was then administered a daily nutritional composition supplement comprising 11 grams of omega-6 and 1.2 grams of omega-3, made up primarily from a combination of vegetable oils, nuts and seeds which supplied effective amounts of phytochemicals. Administration of the lipid-containing nutritional composition resulted in a reduction of LDL from 160 mg to 120 mg. Very low levels of blood pressure were observed, 90/55 mmHg, when omega-3 was increased to 1.8 grams; blood pressure levels normalized at 105/70 mmHg at 11 grams of omega-6 and 1.2 grams of omega-3. When omega-3 was reduced from 1.8 grams to 1.2 grams per day, the subject experienced an irregular heartbeat, which subsided over a period of 2-3 weeks. However, when omega-3 was further reduced to 0.5 grams per day, it resulted in an ongoing arrhythmia. This demonstrated that supplementation with phytonutrients derived from vegetable oils, nuts and seeds, wherein the omega-6 to omega-3 ratio was about 9:1, resulted in a significant decrease in LDL cholesterol blood levels (dyslipidemia which is associated with atherosclerosis). This case study also demonstrated that the nutritional compositions and ratios described herein may be useful in moderating blood pressure and arrhythmia.

In another human subject, intense muscle spasms arising from the left thoracic cavity/wall were observed upon withdrawal of habitual coumarin consumption from asafetida. It is hypothesized, that sudden withdrawal of phytochemicals, particularly ones that have blood thinning effect may be harmful.

### Example 7

### Case Study on Mood Swing, Mental Function

The subject host was placed on a trial of varying ratios of omega-6 and omega-3 using various oils and nut combinations. Each time omega-3 was reduced or omega-6 was increased the subject became depressed and was given to crying at the slightest provocation. When omega-3 was increased, it elevated the subject's mood, immediately noticeable. However, within certain ranges of omega-6 and omega-3, the effect was self-adjusting, e.g., over a period of 3-6 weeks the moods normalized. It was also observed that within that range of omega-6 and omega-3, over a period of 3-6 weeks the subject in fact was more grounded at higher levels of omega-6; and was euphoric at higher levels of omega-3. Omega-3 increase enhanced cognitive function, which was immediately noticeable. Omega-3 reduction caused confusion, dyslexia, and a decline in cognitive function but these symptoms subsided with time, again within certain omega-6 and omega-3 ranges. The subject also displayed greater attention span and concentration after omega-6 and omega-3 were optimized over a period of 3-6 weeks, with greater reading speeds and comprehension. Thus, the subject performed better at a lower level of omega-3, which suggests that an adaptation mechanism was activated to compensate for the required level of omega-6 metabolites. There may be a similar adaptation mechanism for required level of omega-3. The cumulative effects of such adaptations could pose a threat to the individual. Since phytochemicals have a significant role in this equation, steady delivery of phytochemicals may also be critical.

### Example 8

### Case Studies on Neural Disorders

### 1. Progressive Supra-Nuclear Palsy

The subject host was a 50-year old woman whose symptoms included dental sensitivity, deteriorating muscle mass, occasional breathing difficulty, easy bruising, mild arrhythmia, and difficult bowel movement. A dentist, as a solution to her sensitive teeth, had extracted and replaced her teeth with dentures at 50. Each of her other symptoms was treated as a stand-alone symptom and treated with non-lipid medications. At 60 she developed loss of balance, diplopia (double vision), and slurry speech. Eventually when she started having bone-shattering falls, she was diagnosed with Progressive Supra-nuclear Palsy (PSP), a neurological disease mainly characterized by loss of neural tissue in the brainstem. The subject then lost ambulation and speech, and developed dysphagia. She passed away at 67 from pneumonia.

The woman had had four healthy deliveries, a healthy life until 50, and had no incidence of neural disease in her family. Closer examination of changes in her life around 50 revealed that around that time the fats in her diet had been significantly cut back because of the prevalent doctrine in the 1980s that fats cause heart-disease, and that all fats are deleterious. Both of the woman's parents in their early 70s, and a brother at 48, had died of heart attacks. Hence, the fat reduction was a precautionary measure to avoid cardiac disease, which was then believed to have a strong genetic component. However, it is hypothesized in the present disclosure that the fats were cut to a point where she became severely deficient in both omega-6, and omega-3 fatty acids. The woman was a postmenopausal vegetarian with high antioxidant and phytochemical intake, and the little fat that was in her diet was either saturated fat (less than 20% of total fat) or monounsaturated fat (70-90% of total fat), mostly olive oil following the then doctrine that held olive oil above all others. Olive oil is 75% monounsaturated oil and rich in polyphenols. Since all fatty acids compete for the same enzymes in the metabolic pathway and antioxidants and phytochemicals increase the requirement for omega-6, in her case the deficiency of omega-6 acid appeared to be the culprit. The deficiency of omega-6 is also evident from her early symptoms: muscle mass requires a balance of omega-6 and omega-3, lack of omega-6-derivative leukotrienes would lead to asthma-like breathing issues (conversely excessive leukotrienes can also lead to asthma like symptoms), deficiency of omega-3 has been linked with arrhythmia, and deficiency of omega-6 derived thromboxanes would lead to easy bruising, and lack of omega-6 derived prostaglandins will impede smooth muscle activity and therefore the bowel movement. The fact that she was post-menopausal made the requirement of omega-6 and omega-3 more critical, since estrogen and androgens, as hypothesized in the present disclosure, have similar actions and benefits as polyunsaturated fats. When the reproductive hormones decline, the body increasingly depends on omega-6 and omega-3 for the physiological functions. Excess phytochemicals, particularly polyphenols also may have contributed to the illness.

### 2. Amyotrophic Lateral Sclerosis

The subject was a vegetarian woman in her mid-30s, on a low fat diet using primarily olive oil and nuts. She had developed Amyotrophic Lateral Sclerosis (ALS)-like symptoms: muscle weakness in hands, arms, legs, and the muscles of speech, twitching and cramping of muscles, shortness of breath, and difficulty in swallowing. The left side of her body was affected more than the right side. Upon administration of a nutritional composition and changes in diet plan that increased omega-6 to about 12 grams, her symptoms disappeared and the muscle tone improved, better than before the onset of symptoms. It is hypothesized that in this instance, the amount of omega-3 relative to omega-6 in the tissue had exceeded the ratio tolerated by the body. Since the vegetarian diet and nuts contributed plenty of antioxidants and phytochemicals, the subject became deficient in omega-6, despite moderate levels of omega-3. The symptoms could be reversed by increase in omega-6 and/or withdrawal of nuts and seeds, and certain phytochemicals.

### Example 9

### Case Study on Weight Gain, Obesity

In a vegetarian host subject it was discovered that there was a band of optimal quantity and ratio of omega-6 and omega-3, beyond which the subject gained weight. At omega-6 of 11 grams and omega-3 of 2 grams, the subject was at 134 lbs. When the inventor gradually reduced omega-3 to 1.2 grams, the subject initially gained 6 lbs., and then after 6 weeks, lost 12 lbs. for an ending weight of 128 lbs. Obesity often has been linked to slow metabolism. In turn, metabolic rate has been linked to cell-membrane composition. High polyunsaturated membrane composition may be linked with fast membrane associated processes. Membrane composition influences all aspects of the energy balance equation: electrolyte gradient balance, neuropeptide regulation, gene regulation and glucose regulation.

### Example 10

### Case Study on Digestive System Disorders

In the host subject, incidences of acid reflux disease, irritable bowels, indigestion, and dyspepsia were observed. Each time omega-6 was increased or omega-3 was decreased the following symptoms appeared: stomach pain, bloating, heartburn, nausea (upset stomach), and burping; but they all disappeared as the body adjusted to increased omega-6. Omega-6 was tested up to 11 grams. It is hypothesized that beyond that point in the particular host the symptoms would persist. Increasing omega-3 beyond 2 grams caused tight dark pellet-like stools. In the optimal omega-6 and omega-3 balance, bile production was optimal as determined by the yellowish brown color of the stools. It was also observed that mucus production in the alimentary canal was optimal with the proper omega-6 and omega-3 quantities and ratio, using mucus production in the oral cavity as an indicator. Halitosis was also observed with 2 grams of omega-3, and got worse when omega-3 was reduced, and then normalized over a period of 3-6 weeks. Arachidonic acid plays a pivotal role in protection and integrity of the intestinal mucosa. Excessive omega-3 can displace arachidonic acid leading to gastro-intestinal mucosal damage.

### Example 11

### Case Study on Ovulation, Reproductive Disorders

In a host subject, a 35-year old female, cessation of ovulation (as indicated by watery pale menstrual cycles), intense ovulation-related pains and anovulatory menstruation at extremely low omega-6 in diet were observed; olive oil being the main fat source. It is hypothesized herein that this was due to deficiency of omega-6 derived prostaglandins, which aid ovulation. The same phenomenon was observed when the subject was put on Advil, which blocks cyclooxygenase activity and therefore the prostaglandin synthesis.

### Example 12

### Case Study on Dental Diseases

In a vegetarian host subject, less dental sensitivity, reversal of gum receding, brightening of tooth enamel, and lessening of dental spots and plaque may be exhibited when omega-3 was reduced from 2 grams to 1.2 grams while holding omega-6 constant at 11 grams. Dietary compositions comprising nuts and oils were the source of phytochemicals, omega-6 and omega-3 fatty acids. There was an adjustment period of 3-6 weeks, when the symptoms got worse in the host subjects before getting better. Longer-term intervention studies should be able to test a hypothesis by studying tooth loss during the intervention period. Bioactivity of lipids may explain the linkage between periodontitis/tooth loss and coronary heart disease.

### Example 13

### Case Study on Myofascial Pains and Thoracic Outlet Syndrome

In a 35-year old vegetarian female, on a low-fat diet using olive oil as the main fat in the diet, the development of episodes of acute myofascial pains were observed. The subject experienced severe muscle tightness in several areas of the body, neck shoulders, para-spinal muscles, thighs, hands, and arms.

The host was diagnosed with Myofascial Pain Syndrome (MFS) and Thoracic Outlet Syndrome (TOS). TOS consists of a group of distinct disorders that affect the nerves in the brachial plexus (nerves that pass into the arms from the neck) and the subclavian artery and vein blood vessels between the base of the neck and axilla (armpit). For the most part, these disorders are produced by compression of the components of the brachial plexus (the large cluster of nerves that pass from the neck to the arm), the subclavian artery, or the subclavian vein. Neurogenic form of TOS accounts for 95-98% of all cases of TOS, hence neural disease was suspected. The host subject went through numerous examinations including: MRIs of the entire CNS, X-rays, blood work, drug therapies, massage therapies, and chiropractic treatment. The symptoms would go away and then reappear a few months or a year later. It was observed that the symptoms appeared upon increase of omega-6 fatty acids and/or saturated fatty acids, and/or withdrawal of certain plant matter, for example celery and brown rice. After fatty acids in the subject's diet were optimized by administration of the disclosed lipid compositions, the episodes of TOS and myofascial pains subsided. It is hypothesized herein that these episodes were the result of the body being severely deficient in certain fatty acid metabolites. Each time there was an inadvertent increase in fatty acids, more particularly omega-6 fatty acids and/or their metabolites, which can occur by any incidental changes in diet/withdrawal of a modulating nutrient, there may have been a sudden surge in prostaglandins, thromboxanes, and leukotrienes, and excitability of neural and muscle cells, resulting in severe muscular tightening. Other mechanisms related to the lipids may be involved that are not yet understood.

### Example 14

### Case Studies on Immunity, Autoimmune and Infectious and Inflammatory Diseases

In a vegetarian host subject, a 48-year old menopausal woman, on 11 g of LA and 1.8 g of ALA, from oils and nuts, spinal burning sensation, heat in the body, skin and feet, and delayed wound healing were observed. The subject also developed vaginal yeast infection. Symptoms disappeared upon reducing ALA to 1.2 g after an initial adjustment period. It is hypothesized that omega-6 and omega-3 and plant matter imbalance leads to inflammation, compromised immunity, and infection. It is further suspected that both omega-6 and omega-3 are anti-inflammatory in small doses and inflammatory in large doses.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1-52. (canceled)

53. A method for selecting a nutritional formulation or plan for an individual, comprising:
determining for the individual a diet cohort, the cohort being high plant food, high meat, or high seafood; and
supplementing the individual's diet with one or more nutritional modules comprising one or more of natural oils, butters, margarines, nuts, seeds, herbs, lipids, phytochemicals, antioxidants, vitamins, and minerals, so as to balance the individual's nutritional state.

54. The method of claim 53, which involves use of a kit comprising the formulation or modules, wherein one or more of the following apply:
(i) the kit comprises individual portions of food items for daily consumption;
(ii) the kit comprises individual portions of food items for supplementation of daily diet of a subject;
(iii) the kit comprises a label comprising at least one indication of the suitability of the modules or packages for a consumer with a specific dietary profile or cohort;
(iv) the kit comprises an indication of the upper limit of average daily consumption of items in the kit or module; or
(v) a label is attached to the packaging of the kit or module.

55. The method of claim 53, wherein the individual has signs or symptoms of a chronic disease.

56. A nutritional formulation for an individual comprising at least one module for consumption by a consumer, the formulation comprising:
one or more nutrients in each module,
wherein the nutritional formulation comprises amounts and types of phytochemicals, antioxidants, vitamins, minerals, acid-base, lipids, proteins, carbohydrates, probiotics, prebiotics, microorganisms, fiber and other nutrients that are optimized and balanced to provide a health benefit when one or more servings of the nutritional formulation is used to provide at least 25% of the average daily calories to the consumer over one or more weeks.

57. The formulation of claim 56, wherein determination of the nutritional formulation comprises one or more of the following:
(i) determining a cohort of the individual based on primary dietary ingredients of the subject's daily or weekly diet by comparing levels of one or more of antioxidants, phytochemicals, vitamins, minerals, lipids, carbohydrates, and proteins from foods comprising the subject's diet with levels in a set of predetermined cohorts;
(ii) determining a cohort based on average daily consumption of one or more of grains, vegetables, fruits, legumes, dairy, meats, seafood, herbs, sweeteners, and beverages;
(iii) selecting a cohort from vegetable-based, meat-based and seafood-based; or
(iv) selecting a cohort based on gender, age, genetic profile, family history, climactic temperature, or medical condition.

58. The formulation of claim 56, wherein the modules comprise one or more of:
(i) food items sufficient to supplement the consumer's diet and/or one or more nutrients selected to supplement a cohort;
(ii) less than 500 calories or 25% of daily calories;
(iii) vegetable or vegetable juice packs, fruit or fruit juice packs, dry grain packs, cereal packs, legume, grain, nuts, seeds packs, meat and/or seafood packs, herbs, lipids, meals, snack, side dish, salad, desserts, milks, powder, puree, and/or yogurt;
(iv) nutrients selected from phytochemicals, lipids, antioxidants, vitamins, minerals, synbiotics, probiotics, prebiotics, microorganisms and fiber;
(v) whole food items from natural sources;
(vi) natural sources of lipids selected from oils, butters, margarines, nuts, and seeds;
(vii) micronutrients derived entirely or partly from natural sources; or
(viii) liquid, cream, or patch for topical use.

59. The formulation of claim 56, wherein the modules comprise one or more of:
(i) a part or entire daily dietary intake of nutrients for the subject;
(ii) supplements, balances or replaces the subject's daily food consumption based on the subject's cohort or the subject's lipid consumption;
(iii) at least 80% of daily or weekly total caloric intake for the subject; or
(iv) suit satiety and dietary preference of the subject.

60. The formulation of claim 56, wherein food items are selected based on the methods of processing employed to prepare the food item and wherein optionally the processing is selected from hulling, removing a layer, drying, providing fresh, roasting, and grilling.

61. The formulation of claim 56, wherein one or more of the following apply:
(i) the omega-6 to omega-3 fatty acids ratio is greater than 1:1, or greater than 5:1, or greater than 10:1;
(ii) the omega-9 to omega-6 fatty acids ratio is less than 4:1;
(iii) the monounsaturated to polyunsaturated fatty acids ratio is less than 4:1;
(iv) the omega-9 fatty acids are less than 60% or less than 50% of the total lipids;
(v) the omega-6 fatty acids are greater than 20% or greater than 30% of the total lipids;
(vi) the omega-3 fatty acids are less than 20% or less than 10% of the total lipids;
(vii) the omega-6 fatty acids are less than 40 g or less than 25 g; or
(viii) the omega-3 fatty acids are less than 2 g or less than 1 g.

62. The formulation of claim 56, which involves use of a kit comprising the formulation, modules or packages of food items, wherein one or more of the following apply:
(i) the kit comprises individual portions of food items for daily consumption;
(ii) the kit comprises individual portions of food items for supplementation of daily diet of a subject;
(iii) the kit comprises a label comprising at least one indication of the suitability of the modules or packages for a consumer with a specific dietary profile or cohort;
(iv) the kit comprises an indication of the upper limit of average daily consumption of items in the kit or module; or
(v) a label is attached to the packaging of the kit or module.

63. Use of the formulation of claim 56, wherein the module comprises a medicine for prophylaxis or therapy of a medical condition.

64. Use of the formulation of claim 56, wherein the individual has signs or symptoms of a chronic disease.

65. A formulation of claim 56, for use in the prophylaxis or treatment of a medical condition or disease or to ameliorate symptoms of a medical condition or disease, wherein optionally, the medical condition or disease is selected from menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases.

66. A method of prophylaxis and/or treatment of a medical condition in a subject, comprising administering a formulation of claim 56.

67. The method of claim 66, wherein the medical condition or disease is selected from menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases.

68. A process for developing a nutrient consumption program for an individual, the process comprising:
providing one or more lists of food items or modules comprising nutrients for average daily consumption by a subject,
wherein the food items comprise at least 25% of the subject's average daily caloric intake over at least one week,
wherein the food items further comprise a plurality of nutrients selected from phytochemicals, antioxidants, vitamins, minerals, synbiotics, probiotics, prebiotics, microorganisms and fiber in amounts that optimizes and balances the subject's total dietary intake of the nutrients over an extended period of time such that a beneficial effect is provided to the subject.

69. The process of claim 68, wherein determination of the process for developing a nutrient consumption program comprises one or more of the following:
(i) determining a cohort of the individual based on primary dietary ingredients of the subject's daily or weekly diet by comparing levels of one or more of antioxidants, phytochemicals, vitamins, minerals, lipids, carbohydrates, and proteins from foods comprising the subject's diet with levels in a set of predetermined cohorts;
(ii) determining a cohort based on average daily consumption of one or more of grains, vegetables, fruits, legumes, dairy, meats, seafood, herbs, sweeteners, and beverages;
(iii) selecting a cohort from vegetable-based, meat-based and seafood-based; or
(iv) selecting a cohort based on gender, age, genetic profile, family history, climactic temperature, or medical condition.

70. The process of claim 68, wherein the module or one or more food items comprise one or more of:
(i) food items sufficient to supplement the consumer's diet and/or one or more nutrients selected to supplement a cohort;
(ii) less than 500 calories or 25% of daily calories;
(iii) vegetable or vegetable juice packs, fruit or fruit juice packs, dry grain packs, cereal packs, legume, grain, nuts, seeds packs, meat and/or seafood packs, herbs, lipids, meals, snack, side dish, salad, desserts, milks, powder, puree, and/or yogurt;
(iv) nutrients selected from phytochemicals, lipids, antioxidants, vitamins, minerals, synbiotics, probiotics, prebiotics, microorganisms and fiber;
(v) whole food items from natural sources;
(vi) natural sources of lipids selected from oils, butters, margarines, nuts, and seeds;
(vii) micronutrients derived entirely or partly from natural sources; or
(viii) liquid, cream, or patch for topical use.

71. The process of claim 68, wherein the one or more modules or one or more food items comprise one or more of:
(i) a part or entire daily dietary intake of nutrients for the subject;
(ii) supplements, balances or replaces the subject's daily food consumption based on the subject's cohort or the subject's lipid consumption;
(iii) at least 80% of daily or weekly total caloric intake for the subject; or
(iv) suit satiety and dietary preference of the subject.

72. The process of claim 68, wherein food items are selected based on the methods of processing employed to prepare the food item and wherein optionally the processing is selected from hulling, removing a layer, drying, providing fresh, roasting, and grilling.

73. The process of claim 68, wherein one or more of the following apply:
(i) the omega-6 to omega-3 fatty acids ratio is greater than 1:1, or greater than 5:1, or greater than 10:1;
(ii) the omega-9 to omega-6 fatty acids ratio is less than 4:1;
(iii) the monounsaturated to polyunsaturated fatty acids ratio is less than 4:1;
(iv) the omega-9 fatty acids are less than 60% or less than 50% of the total lipids;
(v) the omega-6 fatty acids are greater than 20% or greater than 30% of the total lipids;
(vi) the omega-3 fatty acids are less than 20% or less than 10% of the total lipids;
(vii) the omega-6 fatty acids are less than 40 g or less than 25 g; or
(viii) the omega-3 fatty acids are less than 2 g or less than 1 g.

74. The process of claim 68, wherein the list provides one or more of the following:
(i) predetermined natural sources of lipids, the sources selected from oils, butters, margarines, nuts and seeds, and optionally one or more of nutrients selected from antioxidants, phytochemicals, vitamins and minerals in amounts that optimizes dietary nutrients such that the subject's lipid intake provides a beneficial effect to the subject;
(ii) a recommendation for consumption of food items over at least one week;
(iii) wherein the food items listed in the nutrient consumption program are optimized to suit satiety and dietary preferences of the subject; or
(iv) the food items that should not be included in the subject's daily diet; should be limited in the subject's daily diet; or should be added to the subject's daily diet.

75. The process of claim 68, which involves use of a kit comprising the formulation, modules or packages of food items, wherein one or more of the following apply:
(i) the kit comprises individual portions of food items for daily consumption;
(ii) the kit comprises individual portions of food items for supplementation of daily diet of a subject;
(iii) the kit comprises a label comprising at least one indication of the suitability of the modules or packages for a consumer with a specific dietary profile or cohort;
(iv) the kit comprises an indication of the upper limit of average daily consumption of items in the kit or module; or
(v) a label is attached to the packaging of the kit or module.

76. A computer system for computationally implementing the process of claim 68, comprising:
(a) a computing device having a memory;
(b) an input device for entering information regarding the subject's actual dietary intake into the memory;
(c) a data base in the memory for storing the information;
(d) a first application program, for execution in the computing device, for determining a dietary cohort of the subject corresponding to the subject's actual dietary intake; wherein optionally the dietary cohort of the subject is
(i) predetermined and entered directly in the computing device; and/or
(ii) determined either manually or computationally; and/or
(iii) selected from vegetable-based, seafood based and meat based;
(e) a nutrient database in the memory of the device for storing dietary guidelines relative to dietary cohorts of a subject; wherein optionally the nutrient database comprises suitable ranges for average daily dietary consumption of nutrients corresponding to each dietary cohort, and/or suitable ranges for daily dietary consumption of carbohydrates, protein, vitamins, minerals and phytochemicals;
(f) a knowledge base in the memory having rules for manipulating the information in the data base to provide a recommended future dietary program for the user, the program comprising one or more of nutrients selected from antioxidants, phytochemicals, phytosterols, vitamins and minerals in amounts that optimize dietary nutrients to provide a beneficial effect to the subject, when at least 25% or optionally at least 70% of the subject's average daily calories are obtained from food listed in the program;
(g) a second application program, for execution in the computing device, for applying the rules in the knowledge base to the information in the data base and to the guidelines in the nutrient base and for generating a nutrition program for the user in a result base; and
(h) means for outputting the contents of the result base, under the direction of the application program,
wherein the nutrition program contents comprise a listing of particular foods suggested for daily consumption by the subject.

77. Use of the process of claim 68, wherein the module comprises a medicine for prophylaxis or therapy of a medical condition.

78. Use of process of claims 68, wherein the individual has signs or symptoms of a chronic disease.

79. A formulation developed according to the process of claim 68, for use in the prophylaxis or treatment of a medical condition or disease or to ameliorate symptoms of a medical condition or disease, wherein optionally, the medical condition or disease is selected from menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases.

80. A method of prophylaxis and/or treatment of a medical condition in a subject, comprising administering a formulation developed according to the process of claim 68.

81. The method of claim 80, wherein the medical condition or disease is selected from menopause, aging, allergy, musculoskeletal disorders, vascular diseases, hypercholesterolemia, mood swing, reduced cognitive function, cancer, neural disorders, mental disorders, renal diseases, endocrine disorders, thyroid disturbances, weight gain, obesity, diabetes, digestive system disorders, reproductive disorders, infant abnormalities, pulmonary disorders, ophthalmologic disorders, dermatological disorders, sleep disorders, dental diseases, autoimmune diseases, infectious diseases, and inflammatory diseases.
